(19) United States
(12) Patent Application Publication
(10) Pub. No.: US 2026/0338993 A1
(43) Pub. Date: Oct. 29, 2026

(54) COMPOSITION, LIGHT ABSORPTION FILTER, OPTICAL FILTER, MANUFACTURING METHOD FOR OPTICAL FILTER, ORGANIC ELECTROLUMINESCENT DISPLAY DEVICE, INORGANIC ELECTROLUMINESCENT DISPLAY DEVICE, LIQUID CRYSTAL DISPLAY DEVICE, AND COMPOUND

(71) Applicant: FUJIFILM Corporation, Tokyo (JP)

(72) Inventors: Masatoshi MIZUMURA, Minamiashigara-shi (JP); Daisuke SASAKI, Minamiashigara-shi (JP); Nobutaka FUKAGAWA, Minamiashigara-shi (JP)

(73) Assignee: FUJIFILM Corporation, Tokyo (JP)

(21) Appl. No.: 19/529,249

(22) Filed: Feb. 4, 2026

Related U.S. Application Data
(63) Continuation of application No. PCT/JP2024/034468, filed on Sep. 26, 2024.

(30) Foreign Application Priority Data
Sep. 29, 2023 (JP) 2023-170302

(51) Int. Cl. G02B 1/04; C08K 5/3432; C08K 5/46; C09B 29/00; C09B 67/20; C09D 5/32; C09D 7/41; C09D 133/06; G02B 5/22

(52) U.S. Cl. CPC G02B 1/04; C09B 29/00; C09B 67/0063; C09D 5/32; C09D 7/41; C09D 133/064; C08K 5/3432; C08K 5/46; G02B 5/22

(57) ABSTRACT

Provided are a composition or a light absorption filter containing a resin, a compound that generates a radical upon ultraviolet irradiation, and an azo-based coloring agent represented by General Formula (i), a compound used in the composition or the light absorption filter, an optical filter using the light absorption filter, a manufacturing method for an optical filter, and an organic electroluminescent display device, an inorganic electroluminescent display device, and a liquid crystal display device.

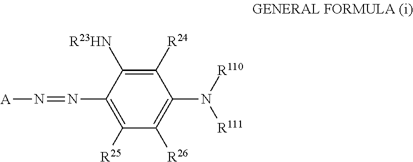

In Formula, R²³ to R²⁶, R¹¹⁰, and R¹¹¹ represent a hydrogen atom or a specific substituent, and A represents a 5- or 6-membered aromatic hydrocarbon group or aromatic heterocyclic group.

### CROSS-REFERENCE TO RELATED APPLICATIONS

This application is a Continuation of PCT International Application No. PCT/JP2024/034468 filed on Sep. 26, 2024, which claims priority under 35 U.S.C. § 119(a) to Japanese Patent Application No. 2023-170302 filed in Japan on Sep. 29, 2023. Each of the above applications is hereby expressly incorporated by reference, in its entirety, into the present application.

### BACKGROUND OF THE INVENTION

### 1. Field of the Invention

The present invention relates to a composition, a light absorption filter, an optical filter, and a manufacturing method for an optical filter, an organic electroluminescent display device, an inorganic electroluminescent display device, a liquid crystal display device, and a compound.

### 2. Description of the Related Art

As an image display device, an organic electroluminescent (OLED) display device, an inorganic electroluminescent display device (inorganic EL display device), a liquid crystal display device, and the like have been mainstream in recent years and are widely used.

A liquid crystal display device is widely used year by year as a space-saving image display device with low power consumption. The liquid crystal display device is a non-light emitting element in which the liquid crystal panel itself displaying an image does not emit light, and thus the liquid crystal display device includes a backlight unit which is disposed on a rear surface of the liquid crystal panel and supplies light to the liquid crystal panel.

The OLED display device is a device that displays an image by utilizing self-luminescence of OLED elements. Therefore, the OLED display device has advantages that a high contrast ratio, a high color reproducibility, a wide viewing angle, a high-speed responsiveness, and a reduction in thickness and weight can be achieved, as compared with various display devices such as a liquid crystal display device and a plasma display device. In addition to these advantages, in terms of flexibility, research and development are being actively carried out as a next-generation display device.

The inorganic EL display device is a device that displays an image by utilizing self-luminescence of inorganic EL elements as a fluorescent material, instead of the OLED elements in the OLED display device. As a result of recent research, it is expected that a display device more excellent than the OLED display device in terms of a large screen size, a longer service life, and the like can be realized.

In the development of the image display device, a technique of incorporating a light absorption filter as a configuration is known.

For example, in a liquid crystal display device, in a case where a white light emitting diode (LED) is used as a light source for a backlight unit, an attempt has been made to provide a light absorption filter in order to block light having unnecessary wavelengths emitted from the white LED.

Regarding another form of the light absorption filter that is incorporated in an image display device, research has been also carried out on an optical filter having both a light absorptive portion having a light absorption effect and a portion in which the light absorbability has been eliminated (hereinafter, also simply referred to as a “light absorption property-eliminated portion”), which is obtained by eliminating the light absorbability of a desired portion.

In particular, in a form in which an optical filter is used by being incorporated in an image display device, light absorption characteristics close to being colorless are required at a light absorption property-eliminated portion in the optical filter.

For example, WO2021/132674A describes a light absorption filter containing a resin; a dye that has a main absorption wavelength band in a wavelength range of 400 to 700 nm; and a compound that generates a radical upon ultraviolet irradiation, in which the dye includes a squaraine-based coloring agent represented by General Formula (1) or a benzylidene-based coloring agent described in WO2021/132674A or a cinnamylidene-based coloring agent represented by General Formula (V). According to the light absorption filter described in WO2021/132674A, it is said that a high decolorization rate is exhibited upon ultraviolet irradiation, and absorption derived from a new coloration structure (hereinafter, also referred to as “secondary absorption”) associated with the decomposition of the dye upon ultraviolet irradiation hardly occurs, whereby a high decolorization property can be obtained.

### SUMMARY OF THE INVENTION

However, although high decolorizing properties are obtained, in a case where the light absorption filter described in WO2021/132674A is used, it is necessary to heat the light absorption filter during ultraviolet irradiation in a case where a benzylidene-based coloring agent represented by General Formula (V) or a cinnamylidene-based coloring agent is used, and improvement has been required in terms of the productivity of the optical filter.

As a result of intensive studies, the present inventors have found that, by configuring the light absorption filter to include an azo-based coloring agent, excellent decolorization properties can be obtained even in a case where the light absorption filter is irradiated with ultraviolet rays at room temperature. On the other hand, it has been found that, in the azo-based coloring agent, a coloring agent decomposition product contained in the light absorption property-eliminated portion obtained by ultraviolet irradiation may undergo a structural change by being exposed to heat, and as a result, secondary light absorption may occur to some extent. Therefore, in a case where the optical filter obtained by irradiating the light absorption filter including the azo-based coloring agent with ultraviolet rays or the image display device including the optical filter is stored or used, there is room for improvement from the viewpoint of suppressing time-dependent coloring due to heat, that is, heat resistance.

That is, an object of the present invention is to provide a composition including a coloring agent, which is decolorized by ultraviolet irradiation and exhibits excellent heat resistance after decolorization.

Another object of the present invention is to provide a light absorption filter that can obtain an optical filter in which a light absorption property-eliminated portion obtained by ultraviolet irradiation exhibits excellent heat resistance.

Another object of the present invention is to provide a compound used in the composition and the light absorption filter.

Further, another object of the present invention is to provide an optical filter using the above-described light absorption filter, where the optical filter includes an optical filter having a light absorptive portion and a light absorption property-eliminated portion exhibiting excellent heat resistance at a desired position, and an OLED display device, an inorganic electroluminescent display device, and a liquid crystal display device, which include this optical filter, as well as a manufacturing method for an optical filter.

As a result of intensive studies in view of the above-described object, the present inventors have found that, by configuring the light absorption filter to include an azo-based coloring agent having a specific structure, an optical filter in which a light absorption property-eliminated portion obtained by ultraviolet irradiation exhibits excellent heat resistance can be obtained. Further studies have been carried out based on these findings, whereby the present invention has been completed.

That is, the above object has been achieved by the following means.
<1>

A composition comprising:
a resin;
a compound that generates a radical upon ultraviolet irradiation; and
an azo-based coloring agent represented by General Formula (i),

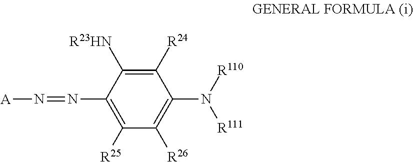

in Formula, R²³ represents a hydrogen atom, —C(═O)R¹⁰¹, —S(═O)₂R¹⁰¹, an acyclic hydrocarbon group, a monocyclic hydrocarbon group, a fused polycyclic hydrocarbon group, or a heterocyclic group,
R¹⁰¹ represents an alkyl group, an alkoxy group, an amino group, or a phenyl group,
R²⁴ to R²⁶ represent a hydrogen atom, a halogen atom, a cyano group, a nitro group, a carboxy group, a sulfo group, —OR¹⁰⁸, —SR¹⁰⁹, —NR¹¹⁰R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, or a heterocyclic group,
R¹⁰⁸ to R¹²¹ represent a hydrogen atom, an acyclic hydrocarbon group, a monocyclic hydrocarbon group, a fused polycyclic hydrocarbon group, or a heterocyclic group, n is a positive integer,
R²⁵ and R²⁶ may be linked to each other to form a hydrocarbon ring, R¹¹⁰ and R¹¹¹ may be linked to each other to form a saturated heterocyclic ring, R²³ and R²⁴ may be linked to each other to form a heterocyclic ring, R²⁴ and R¹¹⁰ may be linked to each other to form a heterocyclic ring, and R²⁶ and R¹¹¹ may be linked to each other to form a heterocyclic ring,
A represents a 5- or 6-membered aromatic hydrocarbon group or aromatic heterocyclic group.
<2>

The composition according to <1>,
in which the compound that generates the radical upon the ultraviolet irradiation includes a combination of
   a compound A having an acid group, and
   a compound B having a structure capable of forming a hydrogen bond with the acid group contained in the compound A.
<3>

The composition according to <2>,
in which the compound A having the acid group is chemically bonded to a polymer that constitutes the resin.
<4>

The composition according to any one of <1> to <3>,
in which the azo-based coloring agent is decolorized by the ultraviolet irradiation.
<5>

A light absorption filter comprising:
a resin;
a compound that generates a radical upon ultraviolet irradiation; and
an azo-based coloring agent represented by General Formula (i),

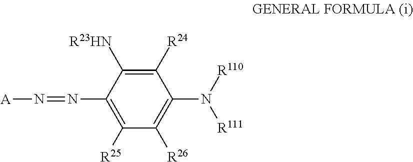

in Formula, R²³ represents a hydrogen atom, —C(═O)R¹⁰¹, —S(═O)₂R¹⁰¹, an acyclic hydrocarbon group, a monocyclic hydrocarbon group, a fused polycyclic hydrocarbon group, or a heterocyclic group,
R¹⁰¹ represents an alkyl group, an alkoxy group, an amino group, or a phenyl group,
R²⁴ to R²⁶ represent a hydrogen atom, a halogen atom, a cyano group, a nitro group, a carboxy group, a sulfo group, —OR¹⁰⁸, —SR¹⁰⁹, —NR¹¹R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, or a heterocyclic group,
R¹⁰⁸ to R¹²¹ represent a hydrogen atom, an acyclic hydrocarbon group, a monocyclic hydrocarbon group, a fused polycyclic hydrocarbon group, or a heterocyclic group, n is a positive integer,
R²⁵ and R²⁶ may be linked to each other to form a hydrocarbon ring, R¹¹⁰ and R¹¹¹ may be linked to each other to form a saturated heterocyclic ring, R²³ and R²⁴ may be linked to each other to form a heterocyclic ring, R²⁴ and R¹¹⁰ may be linked to each other to form a heterocyclic ring, and R²⁶ and R¹¹¹ may be linked to each other to form a heterocyclic ring,
A represents a 5- or 6-membered aromatic hydrocarbon group or aromatic heterocyclic group.
<6>

The light absorption filter according to <5>,
in which the compound that generates the radical upon the ultraviolet irradiation includes a combination of a compound A having an acid group and a compound B having a structure capable of forming a hydrogen bond with the acid group contained in the compound A.
<7>

The light absorption filter according to <6>,
in which the compound A having the acid group is chemically bonded to a polymer that constitutes the resin.
<8>

The light absorption filter according to any one of <5> to <7>,
in which the azo-based coloring agent is decolorized by the ultraviolet irradiation.
<9>

An optical filter obtained by subjecting the light absorption filter according to any one of <5> to <8> to mask exposure by ultraviolet irradiation.
<10>

An organic electroluminescent display device, an inorganic electroluminescent display device, or a liquid crystal display device, comprising:
the optical filter according to <9>.
<11>

The organic electroluminescent display device, the inorganic electroluminescent display device, or the liquid crystal display device according to <10>,
in which a layer that inhibits light absorption of the compound that generates the radical upon the ultraviolet irradiation is provided on a viewer side with respect to the optical filter.
<12>

A manufacturing method for an optical filter, comprising:
irradiating the light absorption filter according to any one of <5> to <8> with an ultraviolet ray to carry out mask exposure.
<13>

A compound represented by General Formula (i-1),

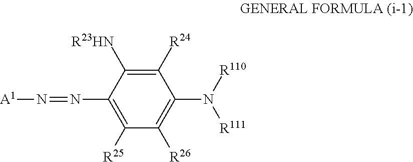

in Formula, R²³ represents a hydrogen atom, —C(═O)R¹⁰¹, —S(═O)₂R¹⁰¹, an acyclic hydrocarbon group, a monocyclic hydrocarbon group, a fused polycyclic hydrocarbon group, or a heterocyclic group,
R¹⁰¹ represents an alkyl group, an alkoxy group, an amino group, or a phenyl group,
R²⁴ to R²⁶ represent a hydrogen atom, a halogen atom, a cyano group, a nitro group, a carboxy group, a sulfo group, —OR¹⁰⁸, —SR¹⁰⁹, —NR¹¹⁰R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, or a heterocyclic group,
R¹⁰⁸ to R¹²¹ represent a hydrogen atom, an acyclic hydrocarbon group, a monocyclic hydrocarbon group, a fused polycyclic hydrocarbon group, or a heterocyclic group, n is a positive integer,
R²⁵ and R²⁶ may be linked to each other to form a hydrocarbon ring, R¹¹⁰ and R¹¹¹ may be linked to each other to form a saturated heterocyclic ring, R²³ and R²⁴ may be linked to each other to form a heterocyclic ring, R²⁴ and R¹¹⁰ may be linked to each other to form a heterocyclic ring, and R²⁶ and R¹¹¹ may be linked to each other to form a heterocyclic ring,
A¹ represents a group represented by any of Formulae (A1) to (A3),

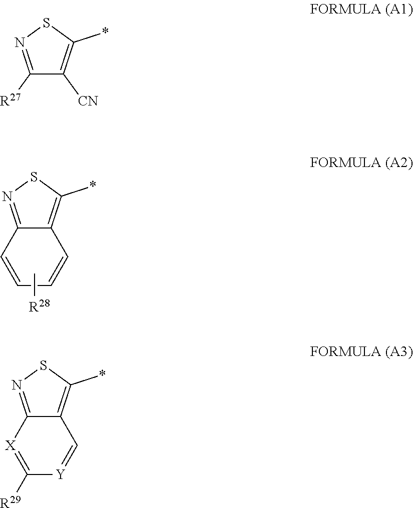

in Formula, R²⁷ represents an unsubstituted methyl group or an aromatic ring group,
R²⁸ represents a hydrogen atom, a methanesulfonyl group, or a sulfo group, or a sulfamoyl group which may be substituted with an alkyl group,
R²⁹ represents an alkyl group, a cyano group, or an alkylsulfanyl group,
X and Y represent CH or a nitrogen atom,
* represents a bonding site.

In the present invention, in a case where there are a plurality of substituents, linking groups, and the like (hereinafter, referred to as substituents and the like) represented by specific reference numerals or formulae, or in a case where a plurality of substituents and the like are defined at the same time, the respective substituents and the like may be the same as or different from each other unless otherwise specified. The same applies to the definition of the number of substituents or the like. In addition, in a case where a plurality of substituents and the like come close to each other (particularly in a case where the substituents and the like are adjacent to each other), the substituents and the like may also be linked to each other to form a ring unless otherwise specified. In addition, unless otherwise specified, rings, for example, alicyclic rings, aromatic rings, and heterocyclic rings may be further fused to form a fused ring.

In the present invention, unless otherwise specified, components constituting the composition and components constituting the light absorption filter (the azo-based coloring agent represented by General Formula (i), the compound that generates a radical upon ultraviolet irradiation, and other components which may be appropriately contained) may be contained in the composition in an amount of one kind or two or more kinds, and may be contained in the light absorption filter in an amount of one kind or two or more kinds. The same applies to an optical filter produced by using the light absorption filter according to the aspect of the present invention.

Unless otherwise specified, the optical filter according to the aspect of the present invention can preferably apply the description regarding the light absorption filter according to the aspect of the present invention, except that it has a light absorption property-eliminated portion formed by ultraviolet irradiation.

In the present invention, in a case where an E type double bond and a Z type double bond are present in a molecule, the double bond may be any one thereof or may be a mixture thereof, unless otherwise specified.

In the present invention, the representation of a compound (including a complex) is used to have a meaning including not only the compound itself but also a salt thereof, and an ion thereof. In addition, it is meant to include those in which a part of the structure is changed within a range where the effect of the present invention is not impaired. Furthermore, it is meant that a compound, which is not specified to be substituted or unsubstituted, may have any substituent within a range where the effect of the present invention is not impaired. The same applies to the definition of a substituent or a linking group.

In addition, in the present invention, the numerical value range indicated by using “to” means a range including the numerical values before and after “to” as the lower limit value and the upper limit value, respectively.

In the present invention, the “composition” includes a mixture in which the component concentration varies within a range in which a desired function is not impaired, in addition to a mixture in which the component concentration is constant (respective components are uniformly dispersed).

In the present invention, the description of “having a main absorption wavelength band in a wavelength range of XX to YY nm” means that a wavelength at which the maximal absorption is exhibited (that is, the maximal absorption wavelength) is present in the wavelength range of XX to YY nm. Therefore, in a case where the maximal absorption wavelength is present in the above-described wavelength range, the entire absorption band including this wavelength may be in the above-described wavelength range or may also extend up to the outside of the above-described wavelength range. In addition, in a case where there are a plurality of maximal absorption wavelengths, it suffices that a maximal absorption wavelength at which the highest absorbance is exhibited is present in the above-described wavelength range. That is, the maximal absorption wavelength other than the maximal absorption wavelength at which the highest absorbance is exhibited may be present either inside or outside the above-described wavelength range of XX to YY nm.

In the present invention, “(meth)acrylate” represents either or both of acrylate and methacrylate, “(meth)acrylic acid” represents any one or both of acrylic acid and methacrylic acid and “(meth)acryloyl” represents any one or both of acryloyl and methacryloyl.

The composition according to the aspect of the present invention includes a coloring agent, and can be decolorized by ultraviolet irradiation and exhibit excellent heat resistance after decolorization.

The light absorption filter according to the aspect of the present invention can obtain an optical filter in which a light absorption property-eliminated portion obtained by ultraviolet irradiation exhibits excellent heat resistance.

In addition, the compound according to the aspect of the present invention is suitably used in the composition and the light absorption filter.

In addition, the optical filter according to the aspect of the present invention can have a light absorptive portion and a light absorption property-eliminated portion exhibiting excellent heat resistance at a desired position.

In addition, the OLED display device, the inorganic electroluminescent display device, and the liquid crystal display device according to the aspect of the present invention comprise the optical filter according to the aspect of the present invention.

The optical filter according to the aspect of the present invention can be preferably manufactured by the manufacturing method according to the aspect of the present invention.

### BRIEF DESCRIPTION OF THE DRAWINGS

FIG. 1 is a schematic view illustrating an outline of one embodiment of a liquid crystal display device having an optical filter according to the present invention.

FIG. 2 is a schematic view showing a simulation configuration regarding the reflection of external light using a light absorption filter according to the present invention.

### DESCRIPTION OF THE PREFERRED EMBODIMENTS

### [Composition and Light Absorption Filter]

The composition according to the embodiment of the present invention contains a resin, a compound that generates a radical upon ultraviolet irradiation, and an azo-based coloring agent represented by General Formula (i).

In the composition according to the embodiment of the present invention, in a case where the compound that generates a radical upon ultraviolet irradiation is dispersed (preferably dissolved) in the resin, the radical is generated by ultraviolet irradiation, the generated radical reacts with the azo-based coloring agent represented by General Formula (i), and the azo-based coloring agent represented by General Formula (i) is chemically changed, typically decomposed, to decolorize the azo-based coloring agent represented by General Formula (i). That is, the azo-based coloring agent represented by General Formula (i) has a characteristic of being chemically changed, typically decomposed, and decolorized by ultraviolet irradiation. The composition according to the embodiment of the present invention can be used without particular limitation, not only for a decolorization application.

Typically, a form of a light absorption filter (light absorption film) is preferably exemplified as the composition according to the embodiment of the present invention.

That is, the light absorption filter according to the embodiment of the present invention contains a resin, a compound that generates a radical upon ultraviolet irradiation, and an azo-based coloring agent represented by General Formula (i).

It is possible to suitably produce the optical filter according to the embodiment of the present invention by subjecting the light absorption filter according to the present invention to mask exposure by ultraviolet irradiation.

The composition and the light absorption filter according to the embodiment of the present invention may contain a dye (coloring agent) other than the azo-based coloring agent represented by General Formula (i). By using the azo-based coloring agent represented by General Formula (i) and the other dye (coloring agent) in combination, a desired color such as black can be adjusted. In addition, by using the azo-based coloring agent represented by General Formula (i) and the other dye (coloring agent) in combination, a change in tint of reflected light can also be suppressed (hereinafter, referred to as “adjusting a tint of reflected light to be neutral”) for a light absorptive portion in the light absorption filter according to the embodiment of the present invention and the optical filter according to the embodiment of the present invention, as compared with a case where the dye is not contained, which is preferable.

As the dye (coloring agent) other than the azo-based coloring agent represented by General Formula (i), which may be contained in the composition and the light absorption filter according to the embodiment of the present invention, any dye (coloring agent) can be used without particular limitation as long as the effect of the present invention is not impaired, and examples thereof include tetraaza porphyrin (TAP)-based, squaraine (SQ)-based, cyanine (CY)-based, benzylidene-based, cinnamylidene-based, azo-based, and indoaniline-based coloring agents (dyes). It is considered that a fading and decolorization mechanism of the dye (coloring agent) other than the azo-based coloring agent represented by General Formula (i) by ultraviolet irradiation occurs in the same manner as the fading and decolorization mechanism of the azo-based coloring agent represented by General Formula (i) by ultraviolet irradiation described above.

In the following description, the term “dye” is used to include a dye (coloring agent) other than the azo-based coloring agent represented by General Formula (i) in addition to the azo-based coloring agent represented by General Formula (i), which may be contained in the composition and the light absorption filter according to the embodiment of the present invention.

In the composition according to the embodiment of the present invention, the “dye” is dispersed (preferably dissolved) in the resin to form a composition exhibiting a specific absorption spectrum derived from the dye. This dispersion may be any type of dispersion, such as a random type or a regular type.

In the light absorption filter according to the embodiment of the present invention, the “dye” is dispersed (preferably dissolved) in the resin to make the light absorption filter a layer that shows a specific absorption spectrum derived from the dye. This dispersion may be any type of dispersion, such as a random type or a regular type.

The composition and the light absorption filter according to the embodiment of the present invention contain a resin, a compound that generates a radical upon ultraviolet irradiation, and an azo-based coloring agent represented by General Formula (i), whereby the composition and the light absorption filter can be decolorized by ultraviolet irradiation and exhibit excellent heat resistance after decolorization.

The reason why the azo-based coloring agent represented by General Formula (i) is decolorized by ultraviolet irradiation is considered as follows based on the results of the studies of the present inventors. In general, the effect of stabilizing a radical by substituting both an electron donating group and an electron withdrawing group with respect to a radical center is known as a “captodative effect”, which is described, for example, on pages 148 to 154 of Acc. Chem. Res., vol. 18 (1985). It is considered that the azo-based coloring agent represented by General Formula (i) has a structure in which an electron-donating group (an amino group represented by —NR¹¹⁰R¹¹¹) is substituted at one terminal of a chromophore, and thus can be decolorized by ultraviolet irradiation by the same concept as the “captodative effect”. In particular, in the azo-based coloring agent represented by General Formula (i), in a case where A is an electron-withdrawing group such as a thiazole group or an isothiazole group, it is considered that an excellent decolorization rate can be obtained even in a case where ultraviolet irradiation is performed at near room temperature (which means 10° C to 37° C) in a mild environment by the “captodative effect”. In addition, since the azo-based coloring agent represented by General Formula (i) itself does not cause almost any secondary absorption due to decomposition of the dye, a portion of the composition and the light absorption filter according to the embodiment of the present invention, which is irradiated with ultraviolet light, can exhibit light transmittance close to colorless.

Furthermore, the reason why the azo-based coloring agent represented by General Formula (i) exhibits excellent heat resistance after decolorization by ultraviolet irradiation is considered as follows, although it is an estimation. It is considered that the azo-based coloring agent represented by General Formula (i) has a group represented by —NHR²³ at an ortho position of an azo group, and a coloring agent decomposition product generated by decolorization of the azo-based coloring agent represented by General Formula (i) by ultraviolet irradiation forms a hydrogen bond with an NH group of the —NHR²³ in the molecule and/or between molecules, whereby the coloring agent decomposition product takes a stable structure. Therefore, the composition and the light absorption filter according to the embodiment of the present invention can exhibit excellent heat resistance even in a case where the composition and the light absorption filter are exposed to heat after decolorization by ultraviolet irradiation, since coloring due to a structural change of the coloring agent decomposition product is suppressed.

In a case where the composition and the light absorption filter according to the embodiment of the present invention contains a compound A having an acid group as will be described later, as a compound that generates a radical upon ultraviolet irradiation, and a compound B having a structure that is capable of forming a hydrogen bond with the acid group contained in the compound A, the efficiency of generating radical species upon ultraviolet irradiation is improved as compared with a case where a commonly used photoradical generator such as a benzophenone compound is used. Therefore, even in a case where the ultraviolet irradiation is carried out under a mild temperature condition such as near room temperature, sufficient radical species are generated, the radical species directly or indirectly react with the dye, and then the dye is decomposed, whereby the dye is faded and decolorized, which makes it possible to exhibit a more excellent decolorization rate.

In addition, in the composition and the light absorption filter according to the embodiment of the present invention, in a case where the compound A having an acid group is bonded to a polymer that constitutes the resin, a radical is generated in the vicinity of the dye upon ultraviolet irradiation, and an effect that the radical easily reacts with the dye is exhibited.

In addition, “the compound A having an acid group” described later may be bonded to a polymer that constitutes the resin. Further, “the compound B having a structure that is capable of forming a hydrogen bond with the acid group contained in the compound A” described above is dispersed (preferably dissolved) in the resin by forming a hydrogen bond with the compound A or forms a hydrogen bond with the compound A in the resin in a case where the compound A containing the acid group is bonded to a polymer that constitutes the resin. In a case of being subjected to ultraviolet irradiation, the compound B generates a radical, and this radical easily reacts with a dye, which makes it possible for a dye to be efficiently faded and decolorized by a mechanism in which the generated radical reacts with a dye in the vicinity.

The azo-based coloring agent represented by General Formula (i) contained in the composition and the light absorption filter according to the embodiment of the present invention may be one kind or two or more kinds.

In addition, in a case where the composition and the light absorption filter according to the embodiment of the present invention contain a dye (coloring agent) other than the azo-based coloring agent represented by General Formula (i), the dye (coloring agent) other than the azo-based coloring agent represented by General Formula (i) may be one kind or two or more kinds.

<Azo-Based Coloring Agent Represented by General Formula (i)>

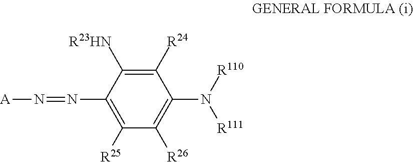

In Formula, R²³ represents a hydrogen atom, —C(═O)R¹⁰¹, —S(═O)₂R¹⁰¹, an acyclic hydrocarbon group, a monocyclic hydrocarbon group, a fused polycyclic hydrocarbon group, or a heterocyclic group.

R¹⁰¹ represents an alkyl group, an alkoxy group, an amino group, or a phenyl group.

R²⁴ to R²⁶ represent a hydrogen atom, a halogen atom, a cyano group, a nitro group, a carboxy group, a sulfo group, —OR¹⁰⁸, —SR¹⁰⁹, —NR¹¹⁰R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, or a heterocyclic group.

R¹⁰⁸ to R¹²¹ represent a hydrogen atom, an acyclic hydrocarbon group, a monocyclic hydrocarbon group, a fused polycyclic hydrocarbon group, or a heterocyclic group. n is a positive integer.

R²⁵ and R²⁶ may be linked to each other to form a hydrocarbon ring, R¹¹⁰ and R¹¹¹ may be linked to each other to form a saturated heterocyclic ring, R²³ and R²⁴ may be linked to each other to form a heterocyclic ring, R²⁴ and R¹¹⁰ may be linked to each other to form a heterocyclic ring, and R²⁶ and R¹¹¹ may be linked to each other to form a heterocyclic ring.

A represents a 5- or 6-membered aromatic hydrocarbon group or aromatic heterocyclic group.

The acyclic hydrocarbon group that can be adopted as R²³ to R²⁶ and R¹⁰⁸ to R¹²¹ means an acyclic alkyl group in which one hydrogen atom is removed from an acyclic alkane, and may be linear or branched. However, the acyclic alkyl group may have a ring structure as a substituent. The number of carbon atoms in the acyclic alkyl group is preferably 1 to 30, more preferably 1 to 20, still more preferably 1 to 12, particularly preferably 1 to 8, and among these, it is preferably 1 to 6. Specific examples thereof include a methyl group, an ethyl group, a propyl group, a butyl group, a sec-butyl group, a tert-butyl group, a pentyl group, a hexyl group, a heptyl group, and an octyl group.

The monocyclic hydrocarbon group that can be adopted as R²³ to R²⁶, and R¹⁰⁸ to R¹²¹ means a monocyclic cycloalkyl group, a monocyclic cycloalkenyl group, a monocyclic cycloalkynyl group, or a monocyclic aryl group, which is a group in which one hydrogen atom is removed from a monocyclic aliphatic hydrocarbon ring (which may be any of a monocyclic cycloalkane, a monocyclic cycloalkene, or a monocyclic cycloalkyne) or a monocyclic aromatic hydrocarbon ring.

The number of carbon atoms in the monocyclic cycloalkyl group, the monocyclic cycloalkenyl group, and the monocyclic cycloalkynyl group is not particularly limited as long as it is allowed in terms of the structure; however, it is more preferably 3 to 30, more preferably 3 to 20, and still more preferably 3 to 16. The number of carbon atoms in the monocyclic aryl group is more preferably 6 to 30, still more preferably 6 to 20, and even still more preferably 6 to 16. Specific examples thereof include a 1,3-cyclohexadienyl group, a 2-cyclopentene-1-yl group, a 2,4-cyclopentadiene-1-ylidene group, and a phenyl group.

The fused polycyclic hydrocarbon group that can be adopted as R²³ to R²⁶, and R¹⁰⁸ to R¹²¹ means a fused polycyclic cycloalkyl group, a fused polycyclic cycloalkenyl group, a fused polycyclic cycloalkynyl group, or a fused polycyclic aryl group, which is a group in which one hydrogen atom is removed from a fused polycyclic aliphatic hydrocarbon ring (which may be any of a fused polycyclic cycloalkane, a fused polycyclic cycloalkene, or a fused polycyclic cycloalkyne) or a fused polycyclic aromatic hydrocarbon ring.

The number of carbon atoms in the fused polycyclic cycloalkyl group, the fused polycyclic cycloalkenyl group, and the fused polycyclic cycloalkynyl group is not particularly limited as long as it is allowed in terms of the structure; however, it is more preferably 8 to 30 and more preferably 8 to 20. The number of carbon atoms in the fused polycyclic aryl group is more preferably 12 to 30 and still more preferably 12 to 20. Specific examples thereof include a pentalenyl group, an indenyl group, a naphthyl group, an azulenyl group, a heptalenyl group, an acenaphthylene group, a phenalenyl group, a fluorenyl group, an anthryl group, a phenanthryl group, a triphenylene group, a pyrenyl group, a chrysene group, a perylene group, a perinyl group, a tetraphenylene group, a hexaphenyl group, a hexaheptalene group, a rubicene group, a coronene group, a trinaphthylene group, a heptaphenyl group, a heptaheneptylene group, a pyraltrene group, and an ovalene group.

The heterocyclic group that can be adopted as R²³ to R²⁶ and R¹⁰⁸ to R¹²¹ may be a saturated or unsaturated aliphatic ring group or an aromatic ring group, but is preferably an aromatic heterocyclic group. Examples of the ring-constituting atom constituting the heterocyclic group include those containing at least any one of a heteroatom such as a nitrogen atom, a sulfur atom, or an oxygen atom, where the heterocyclic group may further have a substituent. The heterocyclic group is preferably a heterocyclic group having a total number of carbon atoms of 1 to 30, and more preferably a heterocyclic group having a total number of carbon atoms of 1 to 15. Specific examples thereof include a thienyl group, a furyl group, a pyrrolyl group, an imidazolyl group, a pyrazolyl group, a pyridyl group, a pyrazinyl group, a pyrimidinyl group, a pyridazinyl group, an indolyl group, a quinolyl group, an isoquinolyl group, a phthalazinyl group, a quinoxalinyl group, a quinazolinyl group, a carbazolyl group, an acridinyl group, a phenazinyl group, a furfuryl group, an isothiazolyl group, an isoxazolyl group, a flurazanyl group, a phenoxazinyl group, a benzothiazolyl group, a benzoxazolyl group, a benzimidazolyl group, a 2-methylpyridyl group, and a 3-cyanopyridyl group.

The halogen atom that can be adopted as R²⁴ to R²⁶ is a fluorine atom, a chlorine atom, a bromine atom, or an iodine atom, and is preferably a chlorine atom.

n is preferably an integer of 1 to 12, more preferably an integer of 1 to 6, and still more preferably an integer of 1 to 3.

The acyclic hydrocarbon group, the monocyclic hydrocarbon group, the fused polycyclic hydrocarbon group, and the heterocyclic group may have one or two or more of a halogen atom, a cyano group, a nitro group, a carboxy group, a sulfo group, —OR¹⁰⁸, —SR¹⁰⁹, —NR¹¹⁰R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, and a heterocyclic group as a substituent, and the number of substituents that may be included is preferably 1 to 3 and more preferably 1 or 2. n and R¹⁰⁸ to R¹²¹ have the same meanings as n and R¹⁰⁸ to R¹²¹ described above.

R²³ is preferably —C(═O)R¹⁰¹, —S(═O)₂R¹⁰¹, an acyclic hydrocarbon group, a monocyclic hydrocarbon group, a fused polycyclic hydrocarbon group, or a heterocyclic group, and more preferably —C(═O)R¹⁰¹.

As the alkyl group that can be adopted as R¹⁰¹, the description of the acyclic alkyl group and the monocyclic cycloalkyl group that can be adopted as R²³ to R²⁶ and R¹⁰⁸ to R¹²¹ described above can be applied, and an acyclic alkyl group is preferable.

Preferred examples of the alkoxy group that can be adopted as R¹⁰¹ include a group in which the alkyl group that can be adopted as R¹⁰¹ is applied as an alkyl group moiety in the alkoxy group.

Examples of the amino group that can be adopted as R¹⁰¹ include an unsubstituted amino group (—NH₂) and a monosubstituted or disubstituted amino group substituted with any of the acyclic hydrocarbon group, the monocyclic hydrocarbon group, the fused polycyclic hydrocarbon group, and the heterocyclic group that can be adopted as R¹¹⁰ and R¹¹¹ described above, and a monosubstituted or disubstituted amino group substituted with an acyclic alkyl group is preferable.

R¹⁰¹ is preferably an alkyl group.

R²⁴ and R²⁵ are preferably a hydrogen atom.

R²⁶ is preferably a hydrogen atom, —OR¹⁰⁸, —SR¹⁰⁹, —NR¹¹R¹¹¹, —NHC(═O)R¹¹⁶, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, or an acyclic hydrocarbon group (preferably an acyclic alkyl group), more preferably a hydrogen atom, —OR¹⁰⁸, —SR¹⁰⁹, or an acyclic alkyl group, and still more preferably a hydrogen atom or an acyclic alkyl group. Here, R¹⁰⁸ to R¹¹¹, R¹¹⁶, and R¹¹⁸ to R¹²¹ are preferably an acyclic alkyl group.

In —NR¹¹⁰R¹¹¹ located at an ortho position with respect to R²⁴ and R²⁶, R¹¹⁰ and R¹¹¹ are preferably an acyclic alkyl group, and more preferably an unsubstituted acyclic alkyl group or an acyclic alkyl group having —OR¹⁰⁸ as a substituent. Here, R¹⁰⁸ is preferably a hydrogen atom or an acyclic alkyl group. In addition, the acyclic alkyl group is preferably unsubstituted with a phenyl group, and more preferably unsubstituted with an aryl group.

R²⁵ and R²⁶ may be linked to each other to form a hydrocarbon ring, R¹¹⁰ and R¹¹¹ may be linked to each other to form a saturated heterocyclic ring, R²³ and R²⁴ may be linked to each other to form a heterocyclic ring, R²⁴ and R¹¹⁰ may be linked to each other to form a heterocyclic ring, and R²⁶ and R¹¹¹ may be linked to each other to form a heterocyclic ring.

The number of rings to be formed may be one or two or more in the compound.

The hydrocarbon ring formed by linking R²⁵ and R²⁶ to each other is preferably a 5- or 6-membered ring and more preferably a 6-membered ring.

The saturated heterocyclic ring formed by linking R¹¹⁰ and R¹¹¹ to each other is preferably a 5- or 6-membered ring and more preferably a 6-membered ring. The saturated heterocyclic ring formed by linking R¹¹⁰ and R¹¹¹ to each other may not contain a heteroatom (preferably an atom selected from a nitrogen atom, an oxygen atom, and a sulfur atom) other than the nitrogen atom to which R¹¹⁰ and R¹¹¹ are bonded, as a ring constituting atom, or may contain the heteroatom. Examples of the saturated heterocyclic ring formed by linking R¹¹⁰ and R¹¹¹ to each other include a piperidine ring, a piperazine ring, a morpholine ring, a thiomorpholine ring, and a piperidone ring, and a morpholine ring is preferable.

The heterocyclic ring formed by linking R²³ and R²⁴ to each other, the heterocyclic ring formed by linking R²⁴ and R¹¹⁰ to each other, and the heterocyclic ring formed by linking R²⁶ and R¹¹¹ to each other are preferably a 5- or 6-membered ring and more preferably a 6-membered ring. The heterocyclic ring formed by linking R²⁴ and R¹¹⁰ to each other and the heterocyclic ring formed by linking R²⁶ and R¹¹¹ to each other may not contain a heteroatom (preferably an atom selected from a nitrogen atom, an oxygen atom, and a sulfur atom) other than the nitrogen atom to which R¹¹⁰ and R¹¹¹ are bonded, as a ring constituting atom, or may contain the heteroatom. In addition, the heterocyclic ring formed by linking R²³ and R²⁴ to each other may not contain a heteroatom (preferably an atom selected from a nitrogen atom, an oxygen atom, and a sulfur atom) other than the nitrogen atom to which R²³ is bonded, as a ring constituting atom, or may contain the heteroatom.

Examples of the heterocyclic ring formed by linking R²³ and R²⁴ to each other, the heterocyclic ring formed by linking R²⁴ and R¹¹⁰ to each other, and the heterocyclic ring formed by linking R²⁶ and R¹¹¹ to each other include a pyrroline ring, a piperidine ring, a tetrahydropyridine ring, a piperazine ring, a morpholine ring, a thiomorpholine ring, a piperidone ring, a triazole ring, and a diazole ring, and a pyrroline ring, a piperidine ring, a tetrahydropyridine ring, or a piperidone ring is preferable.

The hydrocarbon ring formed by linking R²⁵ and R²⁶ to each other, the saturated heterocyclic ring formed by linking R¹¹⁰ and R¹¹¹ to each other, and the heterocyclic ring formed by linking R²⁶ and R¹¹¹ to each other may be unsubstituted or may have a substituent. Examples of the substituent that may be included in these rings include an alkyl group, an oxo group (═O), and an acyl group.

A represents a 5- or 6-membered aromatic hydrocarbon group or aromatic heterocyclic group.

The aromatic hydrocarbon ring constituting the 6-membered ring aromatic hydrocarbon group (hereinafter, simply referred to as “aromatic hydrocarbon group that can be adopted as A”) that can be adopted as A may be a monocyclic ring or a fused ring, each of which is a 6-membered ring. In a case of being a fused ring group, it is preferably a fused ring group of 2 to 6 rings, and the number of ring members of each ring constituting the fused ring group is 6. The number of carbon atoms in the aromatic hydrocarbon group that can be adopted as A is preferably 6 to 30 and more preferably 6 to 16. Specifically, the aromatic hydrocarbon group that can be adopted as A is preferably a phenyl group.

The aromatic heterocyclic group that can be adopted as A (hereinafter, simply referred to as “aromatic heterocyclic group that can be adopted as A”) of a 5- or 6-membered ring may be an aromatic ring group including at least one heteroatom such as a nitrogen atom, a sulfur atom, and an oxygen atom as a ring constituting atom constituting the heterocyclic group.

The aromatic heterocyclic ring constituting the aromatic heterocyclic group that can be adopted as A may be a monocyclic ring or a fused ring. In a case of being a fused ring group, it is preferably a fused ring group of 2 to 6 rings. The number of ring members of each ring constituting the ring and the fused ring group in the monocyclic group may be 5 or 6. The number of carbon atoms in the aromatic heterocyclic group that can be adopted as A is preferably 1 to 25 and more preferably 1 to 15.

Specific examples of the aromatic heterocyclic group that can be adopted as A include a pyrazole group, a 1,2,4-triazole group, an isothiazole group, a benzisothiazole group, a thiazole group, a benzothiazole group, an oxazole group, and a 1,2,4-thiadiazole group.

The aromatic hydrocarbon group that can be adopted as A and the aromatic heterohydrogen group that can be adopted as A may have a substituent. Examples of the substituent which may be contained include a halogen atom, a cyano group, a nitro group, a carboxy group, a sulfo group, —OR¹⁰⁸, —SR¹⁰⁹, —NR¹¹⁰R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, a heterocyclic group, and —S(═O)₂R¹²², and among these, a cyano group, a sulfo group, an alkylsulfanyl group (—SR¹⁰⁹, where R¹⁰⁹ is an alkyl group), a sulfamoyl group which may be substituted with an alkyl group (—S(═O)₂NR¹¹²R¹¹³, where R¹¹² and R¹¹³ are a hydrogen atom or an alkyl group), an alkyl group, an aromatic hydrocarbon group, and an aromatic heterocyclic group are preferable.

R¹²² has the same meaning as R¹⁰⁸ to R¹²¹ described above, and represents a hydrogen atom, an acyclic hydrocarbon group, a monocyclic hydrocarbon group, a fused polycyclic hydrocarbon group, or a heterocyclic group.

In addition, in a case where the aromatic hydrocarbon group that can be adopted as A and the aromatic heterohydrogen group that can be adopted as A have a substituent, adjacent substituents may be bonded to each other to form a ring, and examples of the ring that may be formed include an unsaturated hydrocarbon ring.

From the viewpoint of obtaining an excellent decolorization rate even in a case where ultraviolet irradiation is performed at near room temperature, which is a mild environment, A is preferably an electron-withdrawing group, more preferably an aromatic heterocyclic group of a monocyclic ring or a fused ring having an electron-withdrawing 5- or 6-membered ring as a constitutive ring, still more preferably an aromatic heterocyclic group of a monocyclic ring or a fused ring having an electron-withdrawing 5-membered ring as a constitutive ring, particularly preferably a 1,2,4-triazole group, an isothiazole group, a thiazole group, an oxazole group, a 1,2,4-thiadiazole group, or a benzisothiazole group, and among these, an isothiazole group or a benzisothiazole group is preferable.

Among these, the azo-based coloring agent represented by General Formula (i) is preferably represented by General Formula (i-a).

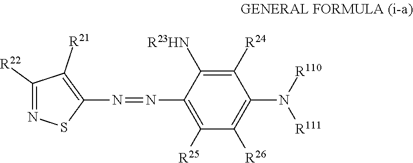

In Formula, R²¹ and R²² represent a hydrogen atom, a halogen atom, a cyano group, a nitro group, a carboxy group, a sulfo group, —OR¹⁰⁸, —SR¹⁰⁹, —NR¹¹⁰R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, or a heterocyclic group.

R²³ to R²⁶, R¹⁰⁸ to R¹²¹, and n have the same meanings as R²³ to R²⁶, R¹⁰⁸ to R¹²¹, and n described above.

R²¹ is preferably a cyano group, and R²² is preferably an alkyl group, an aromatic hydrocarbon group, or an aromatic heterocyclic group.

In addition, it is also preferable that R²¹ and R²² are bonded to each other to form an unsaturated hydrocarbon ring, and it is more preferable that a 5- or 6-membered ring unsaturated hydrocarbon ring is formed. The ring to be formed may have a substituent, and as the substituent which may be contained, the description of the substituent which may be contained in the aromatic hydrocarbon group that can be adopted as A and the aromatic heterohydrogen group that can be adopted as A described above can be applied.

(Compound represented by General Formula (i-1))

In the present invention, as the azo-based coloring agent represented by General Formula (i), a compound represented by General Formula (i-1) is also preferable.

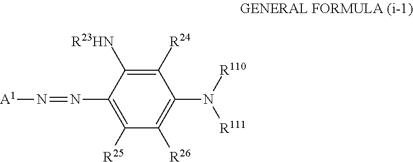

In Formula, R²³ to R²⁶, R¹¹⁰, and R¹¹¹ have the same meanings as R²³ to R²⁶, R¹¹⁰, and R¹¹¹ in General Formula (i) described above.

A¹ represents a group represented by any of Formulae (A1) to (A3).

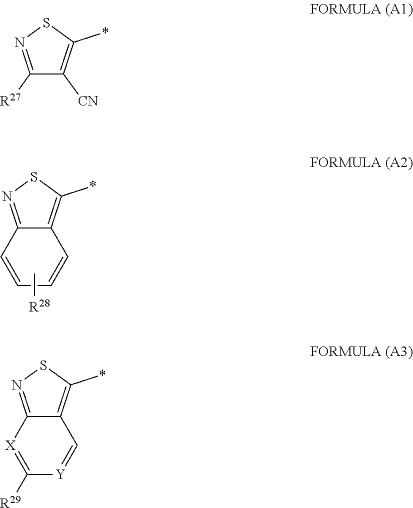

In Formula, R²⁷ represents an unsubstituted methyl group or an aromatic ring group.

R²⁸ represents a hydrogen atom, a methanesulfonyl group, or a sulfo group, or a sulfamoyl group which may be substituted with an alkyl group.

R²⁹ represents an alkyl group, a cyano group, or an alkylsulfanyl group.

X and Y represent CH or a nitrogen atom.

* represents a bonding site.

R²⁷ is preferably an unsubstituted methyl group or an aromatic hydrocarbon group, more preferably an unsubstituted methyl group or a phenyl group, and still more preferably an unsubstituted methyl group or an unsubstituted phenyl group.

R²⁸ is preferably a hydrogen atom, a methanesulfonyl group, —S(═O)₂NH₂, or a dialkylsulfamoyl group, and more preferably a hydrogen atom, a methanesulfonyl group, —S(═O)₂NH₂, or a dimethylsulfamoyl group.

Specific examples of the azo-based coloring agent represented by General Formula (i) include compounds described below, in addition to the compounds used in Examples described later. However, the present invention is not limited thereto.

In the following structural formulae, Me represents a methyl group, Ac represents an acetyl group, Ph represents a phenyl group, n-represents normal, and i-represents iso.

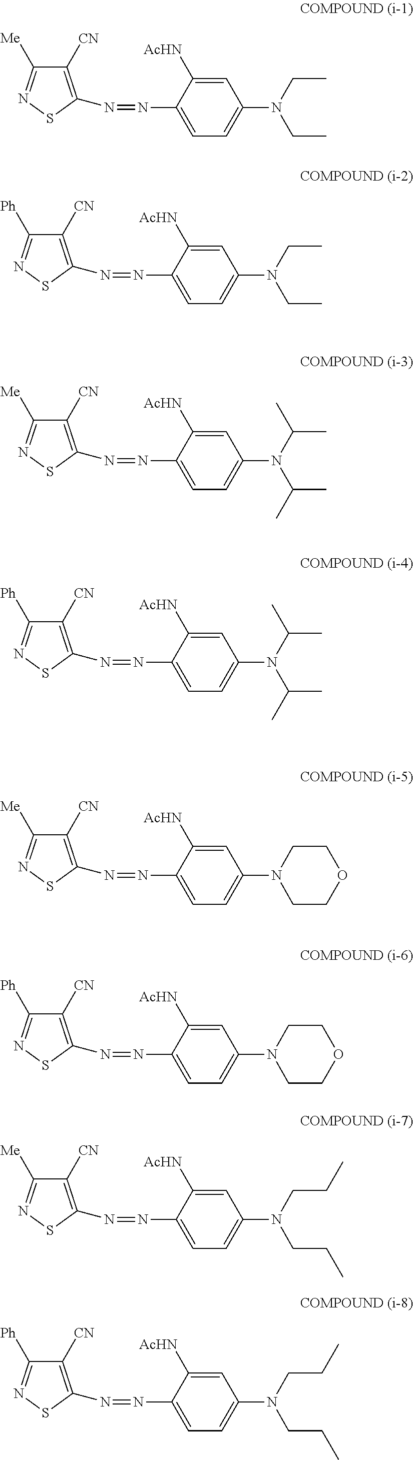

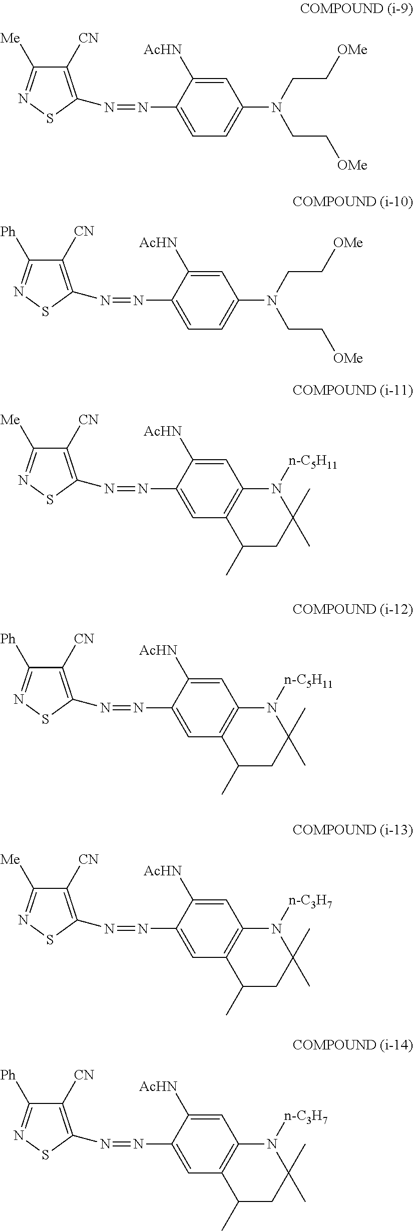

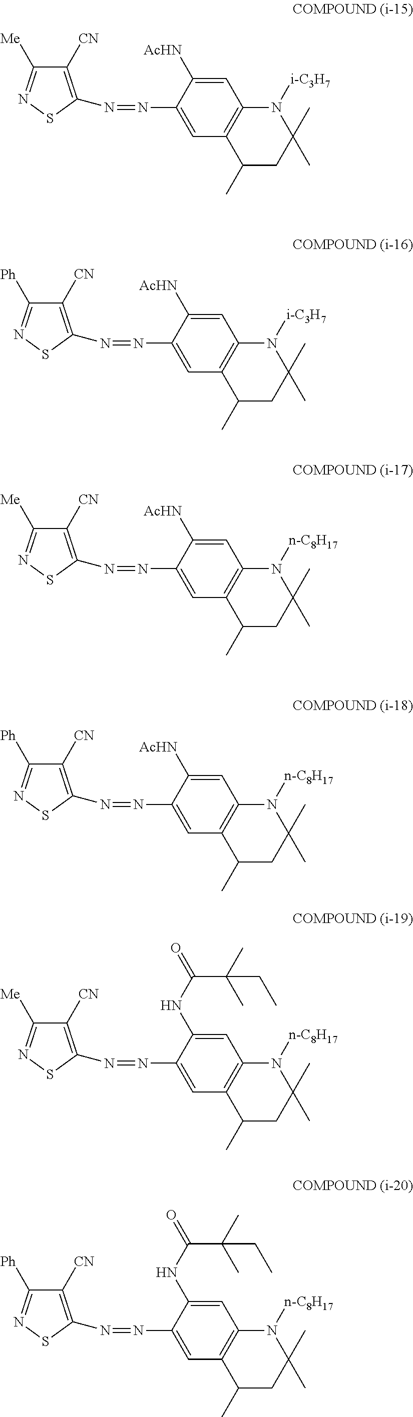

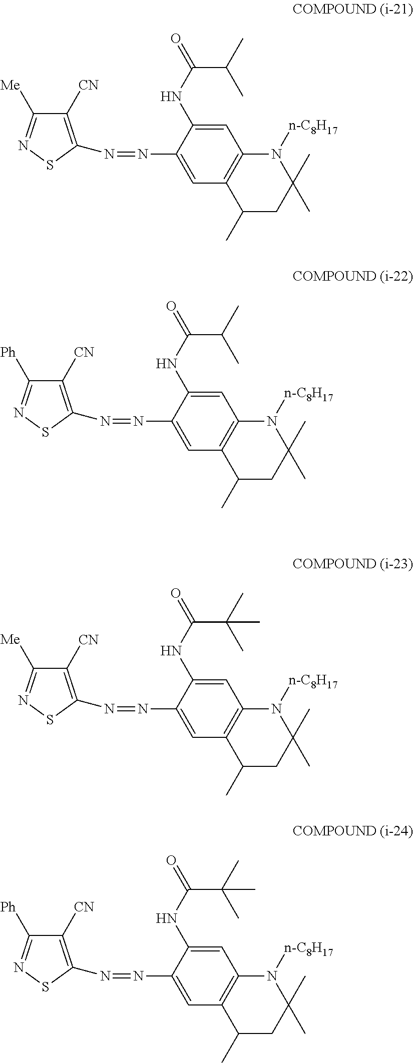

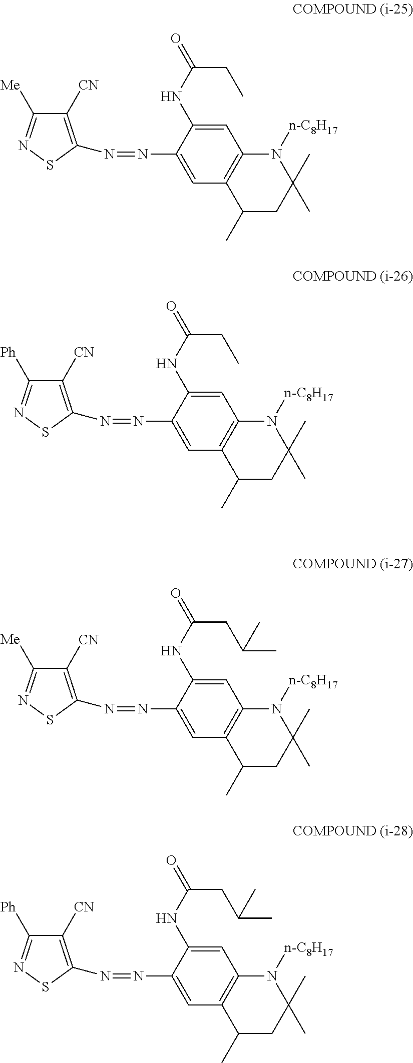

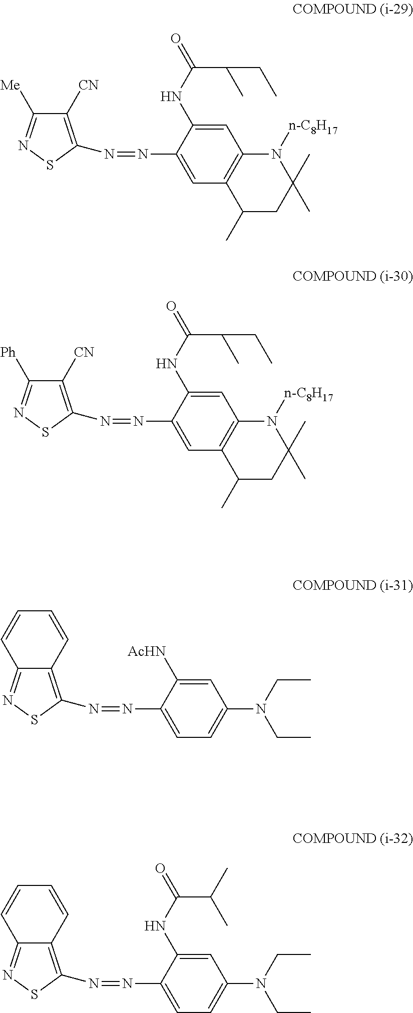

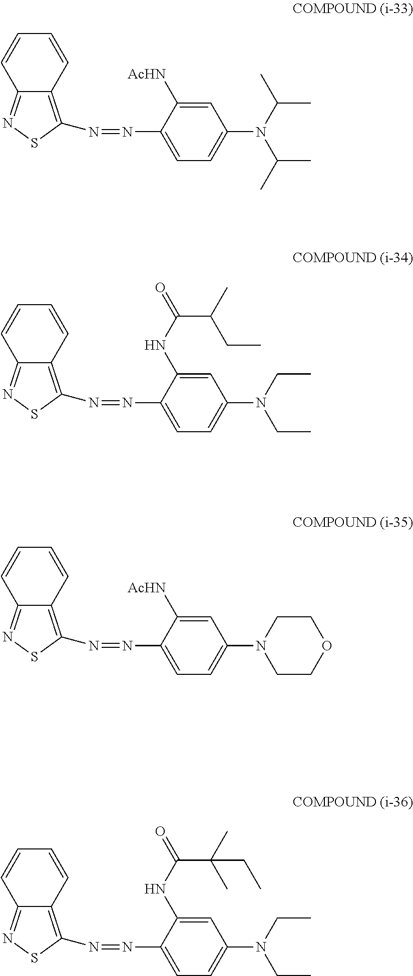 [SMILES: CC(=O)Nc1cc(N(C(C)C)C(C)C)ccc1N=Nc1snc2ccccc12 ; CCC(C)C(=O)Nc1cc(N(CC)CC)ccc1N=Nc1snc2ccccc12 ; CC(=O)Nc1cc(N2CCOCC2)ccc1N=Nc1snc2ccccc12 ; CCN(CC)c1ccc(N=Nc2snc3ccccc23)c(NC(=O)C(C)(C)CC)c1]

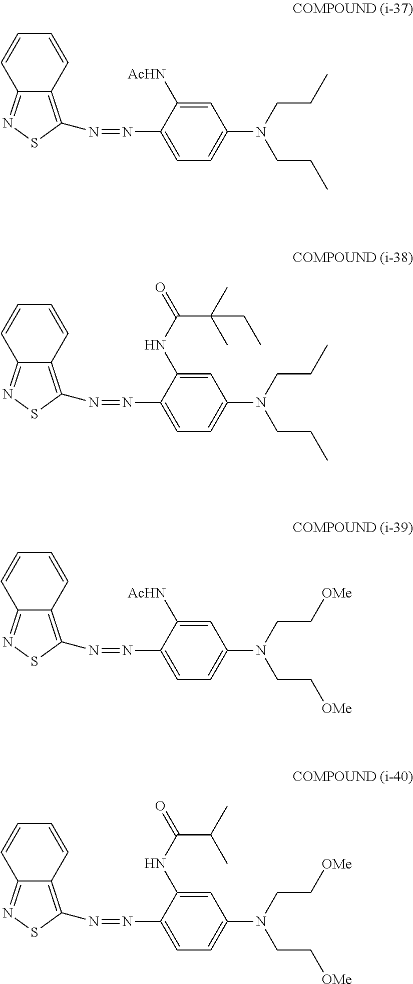 [SMILES: CCCN(CCC)c1ccc(N=Nc2snc3ccccc23)c(NC(C)=O)c1 ; CCCN(CCC)c1ccc(N=Nc2snc3ccccc23)c(NC(=O)C(C)(C)CC)c1 ; COCCN(CCOC)c1ccc(N=Nc2snc3ccccc23)c(NC(C)=O)c1 ; COCCN(CCOC)c1ccc(N=Nc2snc3ccccc23)c(NC(=O)C(C)C)c1]

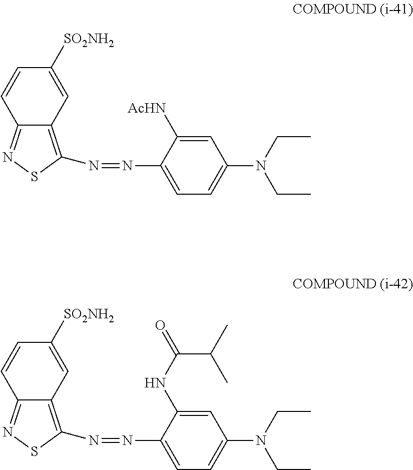 [SMILES: CCN(CC)c1ccc(N=Nc2snc3ccc(S(N)(=O)=O)cc23)c(NC(C)=O)c1 ; CCN(CC)c1ccc(N=Nc2snc3ccc(S(N)(=O)=O)cc23)c(NC(=O)C(C)C)c1]

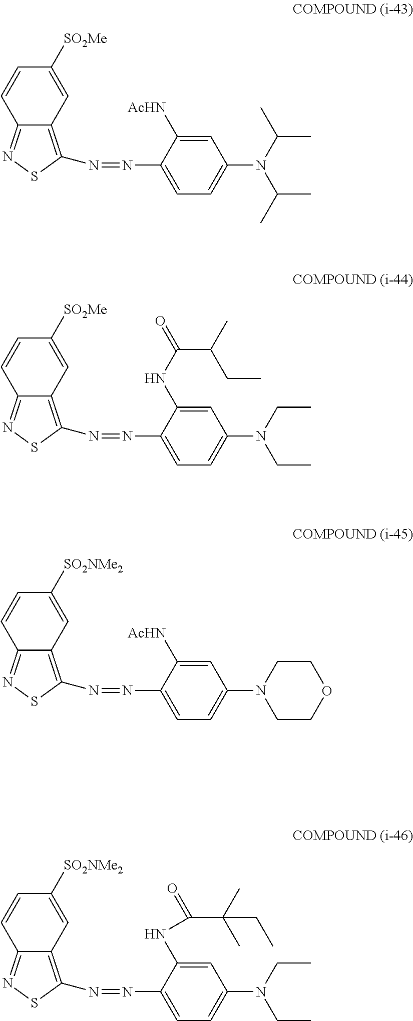

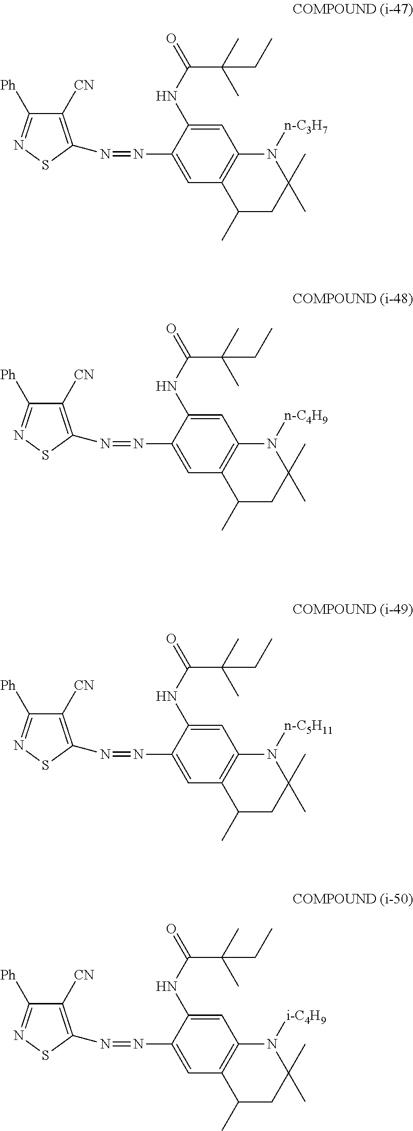

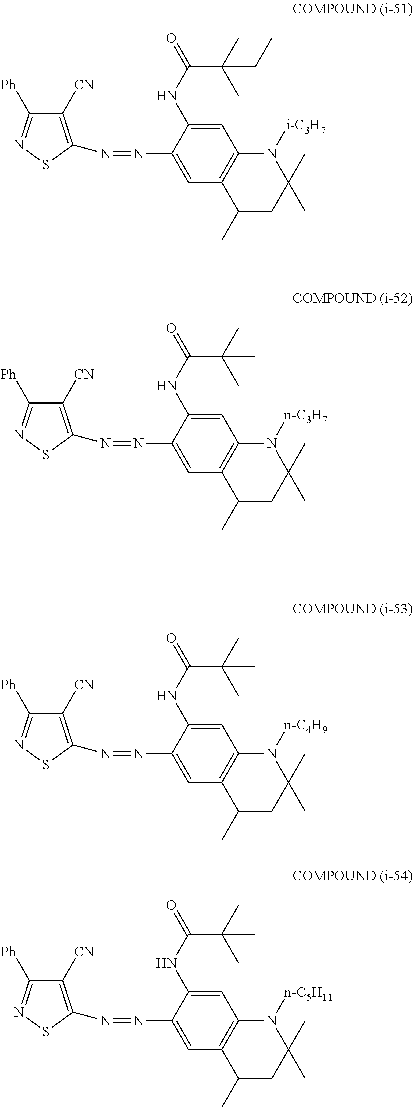

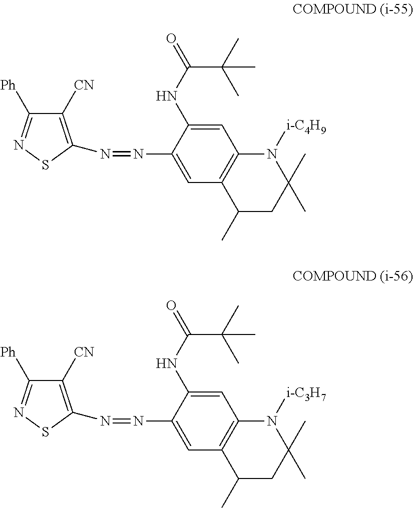

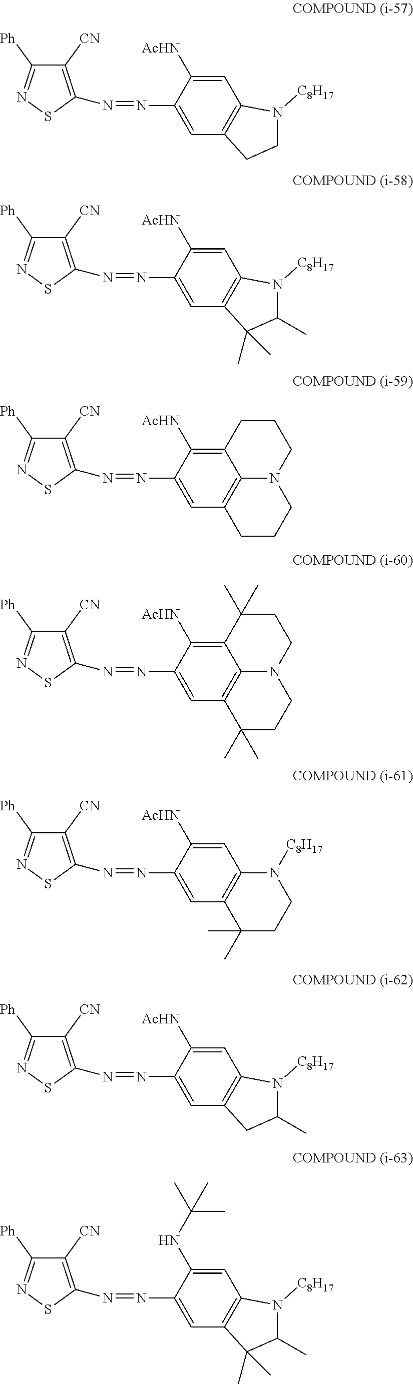

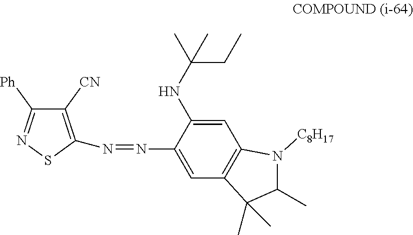

<Azo-Based Coloring Agent Represented by General Formula (ii)>

As the dye (coloring agent) other than the azo-based coloring agent represented by General Formula (i), for example, even in a case of being used in combination with an azo-based coloring agent represented by General Formula (ii), the dye can be decolorized by ultraviolet irradiation and exhibit excellent heat resistance after decolorization.

The azo-based coloring agent represented by General Formula (ii) exhibits excellent decolorization properties because, even in a case where ultraviolet irradiation is performed under mild temperature conditions such as at near room temperature, an excellent decolorization rate can be obtained since a hydroxy group on a pyridine ring bonded to an azo group contributes to the generation of a radical species, and almost no secondary absorption occurs due to decomposition of the dye. As will be described in Comparative Examples later, the azo-based coloring agent represented by General Formula (ii) cannot be decolorized by ultraviolet irradiation and exhibit excellent heat resistance after decolorization in a case of not being used in combination with the azo-based coloring agent represented by General Formula (i).

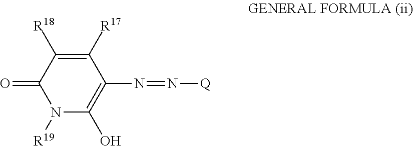

In Formula, R¹⁷ and R¹⁸ each independently represent a hydrogen atom or a monovalent substituent.

R¹⁹ represents a hydrogen atom, an aliphatic group, an aryl group, a heterocyclic group, a carbamoyl group, an alkoxycarbonyl group, an aryloxycarbonyl group, an acyl group, an alkylsulfonyl group, an arylsulfonyl group, or a sulfamoyl group.

Q represents a diazo component residue.

However, R¹⁷ to R¹⁹ and Q do not have a squaraine structure.

The above-described squaraine structure means a structure of a squaraine-based coloring agent. The squaraine-based coloring agent is a coloring agent having a structure that has a skeleton derived from squaric acid in a central part of a R-conjugated system.

Examples of the monovalent substituent that can be adopted as R¹⁷ and R¹⁸ include a halogen atom, an aliphatic group, an aryl group, a heterocyclic group, a cyano group, a carboxy group, a carbamoyl group, an aliphatic oxycarbonyl group, an aryloxycarbonyl group, an acyl group, a hydroxy group, an aliphatic oxy group, an aryloxy group, an acyloxy group, a carbamoyloxy group, a heterocyclic oxy group, an amino group (—NH₂), an aliphatic amino group, an arylamino group, a heterocyclic amino group, an acylamino group, a carbamoylamino group, a sulfamoylamino group, an aliphatic oxycarbonylamino group, an aryloxycarbonylamino group, an aliphatic sulfonylamino group, an arylsulfonylamino group, a nitro group, an aliphatic thio group, an arylthio group, an aliphatic sulfonyl group, an arylsulfonyl group, a sulfamoyl group, a sulfo group, an imide group, and a heterocyclic thio group. Among these, an aliphatic group, an aryl group, a heterocyclic group, a cyano group, a carbamoyl group, an aliphatic oxycarbonyl group, an aryloxycarbonyl group, an acyl group, an aliphatic oxy group, an aryloxy group, an aliphatic amino group, or an arylamino group is preferable mainly from the viewpoint of imparting solubility.

The substituents that can be adopted as R¹⁷ and R¹⁸ may be further substituted.

The aliphatic group that can be adopted as R¹⁷ to R¹⁹ may further have a monovalent substituent, may be saturated or unsaturated, and may be cyclic. Specific examples thereof include an alkyl group, a substituted alkyl group, an alkenyl group, a substituted alkenyl group, an alkynyl group, a substituted alkynyl group, an aralkyl group, and a substituted aralkyl group. The total number of carbon atoms in the aliphatic group is preferably 1 to 30 and more preferably 1 to 16. Specific examples of the aliphatic group include a methyl group, an ethyl group, a butyl group, an isopropyl group, a t-butyl group, a hydroxyethyl group, a methoxyethyl group, a cyanoethyl group, a trifluoromethyl group, a 3-sulfopropyl group, a 4-sulfobutyl group, a 2-(2-hydroxyethoxy)ethyl group, a 2-(2-(acetyloxy)ethoxy)ethyl group, a cyclohexyl group, a benzyl group, a 2-phenethyl group, a vinyl group, and an allyl group.

The aryl group that can be adopted as R¹⁷ to R¹⁹ may further have a monovalent substituent, and the aryl group is preferably an aryl group having a total number of carbon atoms of 6 to 30, and more preferably an aryl group having a total number of carbon atoms of 6 to 16. Specific examples thereof include a phenyl group, a 4-tolyl group, a 4-methoxyphenyl group, a 2-chlorophenyl group, a 3-(3-sulfopropylamino)phenyl group, a 4-sulfamoylphenyl group, a 4-(ethoxyethylsulfamoyl)phenyl group, and a 3-(dimethylcarbamoyl)phenyl group.

The heterocyclic group that can be adopted as R¹⁷ to R¹⁹ may be a saturated or unsaturated aliphatic ring group or may be an aromatic ring group, and it is preferably an aromatic heterocyclic group. Examples of the ring-constituting atom constituting the heterocyclic group include those containing at least any one of a heteroatom such as a nitrogen atom, a sulfur atom, or an oxygen atom, where the heterocyclic group may further have a substituent. The heterocyclic group is preferably a heterocyclic group having a total number of carbon atoms of 1 to 30, and more preferably a heterocyclic group having a total number of carbon atoms of 1 to 15. Specific examples thereof include a 2-pyridyl group, a 2-thienyl group, a 2-thiazolyl group, a 2-benzothiazolyl group, a 2-benzoxazolyl group, and a 2-furyl group.

The carbamoyl group that can be adopted as R¹⁷ to R¹⁹ includes, in addition to an unsubstituted carbamoyl group (—CONH₂), a carbamoyl group substituted with an aliphatic group, an aryl group, or the like.

The carbamoyl group that can be adopted as R¹⁷ to R¹⁹ may further have a monovalent substituent, and the carbamoyl group is preferably a carbamoyl group having a total number of carbon atoms of 1 to 30, and more preferably a carbamoyl group having a total number of carbon atoms of 1 to 16. Specific examples thereof include a methylcarbamoyl group, a dimethylcarbamoyl group, a phenylcarbamoyl group, and an N-methyl-N-phenylcarbamoyl group.

To the aliphatic group in the aliphatic oxycarbonyl group that can be adopted as R¹⁷ and R¹⁸, the description of the aliphatic group that can be adopted as R¹⁷ to R¹⁹ can be applied.

The aliphatic oxycarbonyl group that can be adopted as R¹⁷ and R¹⁸ may further have a monovalent substituent, may be saturated or unsaturated, or may be cyclic, and the aliphatic oxycarbonyl group is preferably an aliphatic oxycarbonyl group having a total number of carbon atoms of 2 to 30, and more preferably an aliphatic oxycarbonyl group having a total number of carbon atoms of 2 to 16. Specific examples thereof include a methoxycarbonyl group, an ethoxycarbonyl group, and a 2-methoxyethoxycarbonyl group.

The alkoxycarbonyl group that can be adopted as R¹⁹ may further have a monovalent substituent, may be saturated or unsaturated, or may be cyclic, and the alkoxycarbonyl group is preferably an alkoxycarbonyl group having a total number of carbon atoms of 2 to 30 and more preferably an alkoxycarbonyl group having a total number of carbon atoms of 2 to 16. Specific examples thereof include a methoxycarbonyl group, an ethoxycarbonyl group, and a 2-methoxyethoxycarbonyl group.

The aryloxycarbonyl group that can be adopted as R¹⁷ to R¹⁹ may further have a monovalent substituent, and it is preferably an aryloxycarbonyl group having a total number of carbon atoms of 7 to 30 and more preferably an aryloxycarbonyl group having a total number of carbon atoms of 7 to 16. Specific examples thereof include a phenoxycarbonyl group, a 4-methylphenoxycarbonyl group, and a 3-chlorophenoxycarbonyl group.

The acyl group that can be adopted as R¹⁷ to R¹⁹ includes an aliphatic carbonyl group, an arylcarbonyl group, and a heterocyclic carbonyl group, where an aspect in which the total number of carbon atoms is 1 to 30 is preferable, and an aspect in which the total number of carbon atoms is 1 to 16 is more preferable. Specific examples thereof include an acetyl group, a methoxyacetyl group, a thienoyl group, and a benzoyl group.

To the aliphatic group in the aliphatic sulfonyl group that can be adopted as R¹⁷ and R¹⁸, the description of the aliphatic group that can be adopted as R¹⁷ to R¹⁹ can be applied.

The aliphatic sulfonyl group that can be adopted as R¹⁷ and R¹⁸ may further have a monovalent substituent, may be saturated or unsaturated, or may be cyclic, where an aspect in which the total number of carbon atoms is 1 to 30 is preferable, and an aspect in which the total number of carbon atoms is 1 to 16 is more preferable. Specific examples thereof include a methanesulfonyl group, a methoxymethanesulfonyl group, and an ethoxyethanesulfonyl group.

The alkylsulfonyl group that can be adopted as R¹⁹ may further have a monovalent substituent, may be saturated or unsaturated, or may be cyclic, where an aspect in which the total number of carbon atoms is 1 to 30 is preferable, and an aspect in which the total number of carbon atoms is 1 to 16 is more preferable. Specific examples thereof include a methanesulfonyl group, a methoxymethanesulfonyl group, and an ethoxyethanesulfonyl group.

The arylsulfonyl group that can be adopted as R¹⁷ to R¹⁹ may further have a monovalent substituent, where an aspect in which the total number of carbon atoms is 6 to 30 is preferable, and an aspect in which the total number of carbon atoms is 6 to 18 is more preferable. Specific examples thereof include a benzenesulfonyl group and a toluenesulfonyl group.

The sulfamoyl group that can be adopted as R¹⁷ to R¹⁹ includes, in addition to an unsubstituted sulfamoyl group (—SO₂NH₂), a sulfamoyl group substituted with an aliphatic group, an aryl group, or the like.

The sulfamoyl group that can be adopted as R¹⁷ to R¹⁹ may further have a monovalent substituent, where an aspect in which the total number of carbon atoms is 0 to 30 is preferable, and an aspect in which the total number of carbon atoms is 0 to 16 is more preferable. Specific examples thereof include an unsubstituted sulfamoyl group, a dimethylsulfamoyl group, and a di-(2-hydroxyethyl)sulfamoyl group.

The imide group that can be adopted as R¹⁷ and R¹⁸ may further have a monovalent substituent, and it is preferably an imide group of a 5- or 6-membered ring. In addition, an aspect in which the total number of carbon atoms in the imide group is 4 to 30 is preferable, and an aspect in which the total number of carbon atoms in the imide group is 4 to 20 is more preferable. Specific examples thereof include a succinimide group and a phthalimide group.

To the aliphatic group in the aliphatic oxy group, the aliphatic amino group, the aliphatic oxycarbonylamino group, the aliphatic sulfonylamino group, and the aliphatic thio group, which can be adopted as R¹⁷ and R¹⁸, the description of the aliphatic group that can be adopted as R¹⁷ to R¹⁹ can be applied.

To the aryl group in the aryloxy group, the arylamino group, the aryloxycarbonylamino group, the arylsulfonylamino group, and the arylthio group, which can be adopted as R¹⁷ and R¹⁸, the description of the aryl group that can be adopted as R¹⁷ to R¹⁹ can be applied.

To the acyl group in the acyloxy group and the acylamino group, which can be adopted as R¹⁷ and R¹⁸, the description of the acyl group, which can be adopted as R¹⁷ to R¹⁹, can be applied.

To the carbamoyl group in the carbamoyloxy group and the carbamoylamino group, which can be adopted as R¹⁷ and R¹⁸, the description of the carbamoyl group, which can be adopted as R¹⁷ to R¹⁹, can be applied.

To the heterocyclic group in the heterocyclic oxy group, the heterocyclic amino group, and the heterocyclic thio group, which can be adopted as R¹⁷ and R¹⁸, the description of the heterocyclic group, which can be adopted as R¹⁷ to R¹⁹, can be applied.

To the sulfamoyl group in the sulfamoylamino group that can be adopted as R¹⁷ and R¹⁸, the description of the sulfamoyl group, which can be adopted as R¹⁷ to R¹⁹, can be applied.

The diazo component residue represented by Q means a residue “Q” of a diazo component “Q-NH₂”. The azo-based coloring agent represented by General Formula (ii) is obtained by a diazo coupling reaction using a diazonium ion “Q-N⁺≡N” obtained from a diazo component “Q-NH₂”. In particular, from the viewpoint of the color reproducibility to be targeted, Q is preferably an aryl group or an aromatic heterocyclic group.

The aromatic hydrocarbon ring constituting the aryl group that can be adopted as Q may be a monocyclic ring or a fused ring, and it is preferably a monocyclic ring. It is preferably an aryl group having a total number of carbon atoms of 6 to 30, and more preferably an aryl group having a total number of carbon atoms of 6 to 16. Specifically, a phenyl group is preferable. The aryl group that can be adopted as Q may have a substituent, and preferred examples of the substituent that may be contained include a sulfamoyl group (preferably an alkylsulfamoyl group or a dialkylsulfamoyl group), a sulfonyl group (preferably an alkylsulfonyl group), and a cyano group.

The aromatic heterocyclic group that can be adopted as Q is an aromatic ring group containing, as a ring-constituting atom constituting a heterocyclic group, at least any one among heteroatoms such as a nitrogen atom, a sulfur atom, and an oxygen atom, where the aromatic heterocyclic group is preferably composed of a 5- or 6-membered ring. The total number of carbon atoms in the aromatic heterocyclic group is preferably 1 to 25 and more preferably 1 to 15. The aromatic heterocyclic ring constituting the aromatic heterocyclic group may be a monocyclic ring or a fused ring, and it is preferably a monocyclic ring.

Specific examples of the aromatic heterocyclic group include a pyrazole group, a 1,2,4-triazole group, an isothiazole group, a benzisothiazole group, a thiazole group, a benzothiazole group, an oxazole group, and a 1,2,4-thiadiazole group.

Examples of the azo-based coloring agent represented by General Formula (ii) include the following exemplary compounds (B-12) to (B-16), (B-18), and (B-19). However, the present invention is not limited thereto.

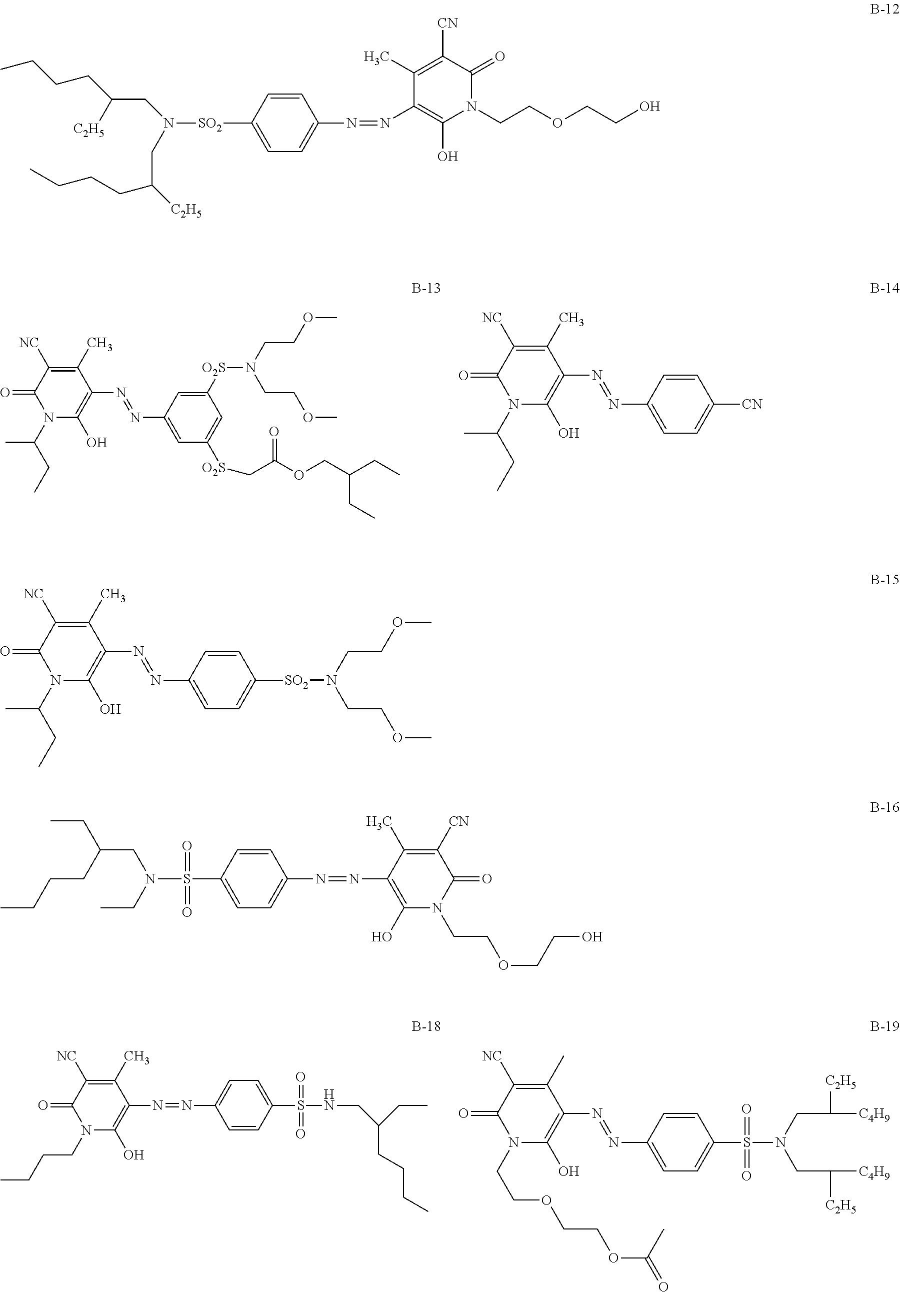

### (Synthesis Method)

The azo-based coloring agent represented by General Formula (i) and the azo-based coloring agent represented by General Formula (ii) can be produced by a conventional method.

For example, the azo-based coloring agent represented by General Formula (i) is obtained by azo coupling of an amino-substituted isothiazole derivative and an amide-substituted aniline derivative shown below. For example, the amino-substituted isothiazole derivative can be synthesized by a method described in JP2009-132639A. In addition, the amide-substituted aniline derivative can be synthesized by, for example, a method described in Journal of Materials Chemistry, 2006, Vol. 16, No. 4, p. 345 to 347. With regard to the conditions for the azo coupling, for example, the conditions described in JP2008-88198A can be referred to. In the synthesis method by the azo coupling, the desired azo-based coloring agent can be obtained by appropriately adjusting the raw material according to the chemical structure of the target azo-based coloring agent represented by General Formula (i) and the azo-based coloring agent represented by General Formula (ii).

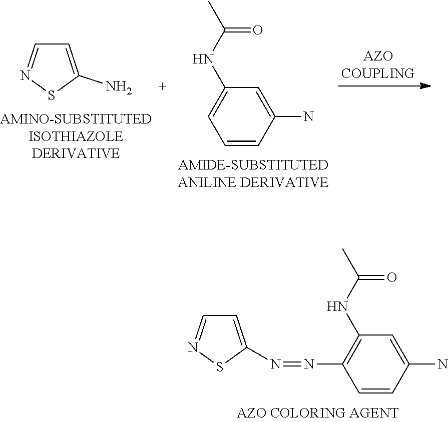

From the viewpoint of obtaining a favorable light absorption property such as an antireflection effect, the total content of the dye in the composition and the light absorption filter according to the embodiment of the present invention is preferably 0.10% to 50% by mass, more preferably 0.15% to 40% by mass, still more preferably 0.20% to 30% by mass, particularly preferably 0.25% to 15% by mass, and especially preferably 0.30% to 10% by mass.

The content of the azo-based coloring agent represented by General Formula (i) in the composition and the light absorption filter according to the embodiment of the present invention is preferably 0.01% to 30% by mass, more preferably 0.1% to 10% by mass, and still more preferably 0.5% to 7.5% by mass.

In a case where the composition and the light absorption filter according to the embodiment of the present invention contain the azo-based coloring agent represented by General Formula (ii), the content of the azo-based coloring agent represented by General Formula (ii) in the composition and the light absorption filter according to the embodiment of the present invention is preferably 0.01% to 30% by mass, more preferably 0.1% to 10% by mass, and still more preferably 0.5% to 7.5% by mass.

In the composition and the light absorption filter according to the embodiment of the present invention, all of the dyes may be the azo-based coloring agent represented by General Formula (i) or may be composed of the azo-based coloring agent represented by General Formula (i) and the azo-based coloring agent represented by General Formula (ii).

<Compound that Generates Radical Upon Ultraviolet Irradiation>

The composition and the light absorption filter according to the embodiment of the present invention contain a compound that generates a radical upon ultraviolet irradiation (hereinafter, also simply referred to as a “radical generator”).

The radical generator is not particularly limited as long as it is a compound that generates a radical upon ultraviolet irradiation, where the compound has a function of decolorizing the above-described dye. As the radical generator, it is possible to use, for example, a photoradical generator which may be used in combination with a compound B, will be described later.

The radical generator also preferably includes a combination of two or more compounds, where the combination is such that two or more compounds interact with each other to form a complex in the composition and the light absorption filter, and as a result, a radical is generated upon ultraviolet irradiation. Regarding the type of the compound to be combined, two or more types of compounds exhibiting functions different from each other need only to be used in a mechanism by which a radical is generated upon ultraviolet irradiation, and the type of the compound to be combined is preferably two types. Preferred examples of such a combination include a combination of a compound A having an acid group and a compound B having a structure that is capable of forming a hydrogen bond with an acid group contained in the compound A.

In a case where the composition and the light absorption filter according to the embodiment of the present invention contains the compound A having an acid group and the compound B having a structure that is capable of forming a hydrogen bond with the acid group contained in the compound A, the efficiency of generating radical species upon ultraviolet irradiation is improved as compared with a case where the photoradical generator is used. As a result, even in a case where the ultraviolet irradiation is carried out under a mild temperature condition such as near room temperature, sufficient radical species are generated, the radical species directly or indirectly react with the dye, and then the dye is decomposed, whereby the dye is faded and decolorized. In particular, the azo-based coloring agent represented by General Formula (i) contained in the composition and the light absorption filter according to the embodiment of the present invention is decolorized without causing almost any secondary absorption due to decomposition of the dye.

Hereinafter, the compound A having an acid group and the compound B having a structure that is capable of forming a hydrogen bond with the acid group contained in the compound A will be described in detail.

### (1) Compound A Having Acid Group

It is preferable that the composition and the light absorption filter according to the embodiment of the present invention contains a compound A having an acid group (also simply referred to as a “compound A” in the present invention) as the radical generator, and a compound B having a structure that is capable of forming a hydrogen bond, together with a compound B having a structure that is capable of forming a hydrogen bond with the acid group contained in the compound A, where the compound B will be described later.

The acid group contained in the compound A is preferably a proton dissociable group having a pKa of 12 or less. Specific examples of the acid group include a carboxy group, a sulfonamide group, a phosphonate group (—P(═O)(OH)₂), a phosphate group (—OP(═O)(OH)₂), a sulfo group, a phenolic hydroxyl group, and a sulfonyl imide group, where a carboxy group is preferable. It is noted that pKa means a negative common logarithm (−log Ka) of the acid dissociation constant (Ka) in water at 25° C, and it can be calculated in the same manner, except that in a calculation of the pKa of the compound B described later, a mixed solvent of water/methanol=50/50 (in terms of volume ratio) is changed to water.

The compound A may be a low-molecular-weight compound or a high-molecular-weight compound (hereinafter, also referred to as a “polymer”), where a polymer is preferable.

That is, the description that the compound A is a polymer means that the compound A is chemically bonded to a polymer that constitutes the resin contained in the composition and the light absorption filter according to the embodiment of the present invention.

In a case where the compound A is a low-molecular-weight compound, the molecular weight of the compound A is less than 5,000, and it is preferably 2,000 or less, more preferably 1,000 or less, still more preferably 500 or less, and particularly preferably 400 or less. The lower limit value thereof is not particularly limited. However, it is practically 100 or more, and it is preferably 200 or more. That is, it is practically 100 or more and less than 5,000, preferably 200 to 2,000, more preferably 200 to 1,000, still more preferably 200 to 500, and particularly preferably 200 to 400.

In a case where the compound A is a polymer, the lower limit value of the weight-average molecular weight of the compound A is 5,000 or more, and it is preferably 10,000 or more and more preferably 15,000 or more from the viewpoint of physical properties of the optical filter. The upper limit value thereof is not particularly limited; however, it is preferably 500,000 or less, more preferably 200,000 or less, and still more preferably 150,000 or less from the viewpoint of solubility in a solvent. That is, 5,000 to 500,000 is practical, 10,000 to 200,000 is preferable, and 15,000 to 150,000 is more preferable.

In addition, a part or all of the acid groups contained in the compound A may or may not be anionized in the composition and the light absorption filter, and in the present invention, both an anionized acid group and a non-anionized acid group are also referred to as an acid group. That is, the compound A may or may not be anionized in the light absorption filter.

The compound A is preferably a compound having a carboxy group from the viewpoint of excellent film-forming properties of the light absorption filter.

The above-described compound having a carboxy group is more preferably a monomer containing a carboxy group (hereinafter, also referred to as a “carboxy group-containing monomer”) or a polymer containing a carboxy group (hereinafter, also referred to as a “carboxy group-containing polymer”), and it is more preferably a carboxy group-containing polymer from the viewpoint of the film-forming properties of the light absorption filter.

It is noted that a part or all of the carboxy groups (—COOH) contained in the carboxy group-containing monomer and the carboxy group-containing polymer may or may not be anionized in the composition and the light absorption filter according to the embodiment of the present invention, and both an anionized carboxy group (—COO⁻) and a non-anionized carboxy group are also referred to as a carboxy group.

That is, the carboxy group-containing polymer may or may not be anionized in the composition and the light absorption filter according to the embodiment of the present invention, and both an anionized carboxy group-containing polymer and a non-anionized carboxy group-containing polymer are also referred to as a polymer.

The content of the compound A in the composition and the light absorption filter according to the embodiment of the present invention is preferably 1% by mass or more, more preferably 25% by mass or more, still more preferably 30% by mass or more, particularly preferably 45% by mass or more, and especially preferably 50% by mass or more. The upper limit value of the content of the compound A is preferably less than 100% by mass, more preferably 99% by mass or less, and still more preferably 97% by mass or less. That is, it is preferably 1% by mass or more and less than 100% by mass, more preferably 25% to 99% by mass, still more preferably 30% to 97% by mass, particularly preferably 45% to 97% by mass, and especially preferably 50% to 97% by mass.

Among the above, in a case where the compound A is a polymer, the content of the compound A in the composition and the light absorption filter according to the embodiment of the present invention is preferably 50% by mass or more and less than 100% by mass, more preferably 60% by mass or more and less than 100% by mass, and still more preferably 70% by mass or more and less than 100% by mass. The upper limit value thereof is also preferably 99% by mass or less, more preferably 97% by mass or less, still more preferably 95% by mass or less, and particularly preferably 90% by mass or less.

One kind of the compound A may be used alone, or two or more kinds thereof may be used in combination.

### (Carboxy Group-Containing Monomer)

Examples of the carboxy group-containing monomer include a polymerizable compound which contains a carboxy group and contains one or more (for example, 1 to 15) ethylenically unsaturated groups.

Examples of the ethylenically unsaturated group include a (meth)acryloyl group, a vinyl group, and a styryl group, and a (meth)acryloyl group is preferable.

It is noted that in a case where the ethylenically unsaturated group is a (meth)acryloyl group, a carbonyl bond in the (meth)acryloyl group and a carbonyl bond in the carboxy group may share one carbonyl bond.

From the viewpoint of more excellent film-forming properties, the carboxy group-containing monomer is preferably a bi- or higher functional monomer containing a carboxy group. The bi- or higher functional monomer means a polymerizable compound having 2 or more (for example, 2 to 15) ethylenically unsaturated groups in one molecule.

It suffices that the number of carboxy groups contained in the carboxy group-containing monomer is 1 or more, and the number thereof is, for example, preferably 1 to 8, more preferably 1 to 4, and still more preferably 1 or 2.

The carboxy group-containing monomer may further have, as an acid group, an acid group other than the carboxy group. Examples of the acid group other than the carboxy group include a phenolic hydroxyl group, a phosphate group, and a sulfonate group.

The bi- or higher functional monomer containing a carboxy group is not particularly limited and can be appropriately selected from known compounds.

Examples of the bi- or higher functional monomer containing a carboxy group include, as product names, ARONIX M-520 and ARONIX M-510 (both manufactured by Toagosei Co., Ltd.).

In addition, examples of the bi- or higher functional monomer containing a carboxy group include a tri- or tetra-functional polymerizable compound having a carboxy group (a compound obtained by introducing a carboxy group into a pentaerythritol triacrylate and pentaerythritol tetraacrylate [PETA] skeleton (acid value=80 to 120 mgKOH/g)) and a penta- or hexa-functional polymerizable compound having a carboxy group (a compound obtained by introducing a carboxy group into a dipentaerythritol pentaacrylate and dipentaerythritol hexaacrylate [DPHA] skeleton (acid value=25 to 70 mgKOH/g)). In a case where the above-described tri- or higher functional monomer containing a carboxy group is used, from the viewpoint of more excellent film-forming properties, it is also preferable to use the bi- or higher functional monomer containing a carboxy group in combination.

Examples of the bi- or higher functional monomer containing a carboxy group and the bi- or higher functional monomer containing an acid group also include the polymerizable compounds having an acid group, which are described in paragraphs 0025 to 0030 of JP2004-239942A. The contents of this patent publication are incorporated in the present specification by reference.

### (Carboxy Group-Containing Polymer)

The carboxy group-containing polymer may further have, as an acid group, an acid group other than the carboxy group. Examples of the acid group other than the carboxy group include a phenolic hydroxyl group, a phosphate group, and a sulfonate group.

In a case where the carboxy group-containing polymer is a copolymer, the structure of the polymer may be a random polymer or a regular polymer such as a block.

### <<Constitutional Unit Having Carboxy Group>>

The carboxy group-containing polymer preferably has a constitutional unit having a carboxy group.

Examples of the constitutional unit having a carboxy group include a constitutional unit derived from (meth)acrylic acid, crotonic acid, itaconic acid, maleic acid, or fumaric acid. Among the above, a constitutional unit derived from (meth)acrylic acid is preferable from the viewpoint of excellent decolorization properties of the dye.

In the carboxy group-containing polymer, a content of the constitutional unit having a carboxy group is preferably 1% to 100% by mole, more preferably 3% to 65% by mole, still more preferably 5% to 45% by mole, and particularly preferably 10% to 45% by mole in a case where the total of all constitutional units of the carboxy group-containing polymer is set to 100% by mole.

One kind of the constitutional unit having a carboxy group may be used alone, or two or more kinds thereof may be used in combination.

### <<Constitutional Unit Having Aromatic Ring>>

It is also preferable that the carboxy group-containing polymer has a constitutional unit having an aromatic ring (preferably, an aromatic hydrocarbon ring) in addition to the above-described constitutional unit. Examples thereof include a constitutional unit derived from a (meth)acrylate having an aromatic ring (specifically, benzyl (meth)acrylate, phenethyl (meth)acrylate, phenoxyethyl (meth)acrylate, or the like).

In the carboxy group-containing polymer, the content of the constitutional unit having an aromatic ring is preferably 0% to 97% by mole, more preferably 0% to 95% by mole, and still more preferably 0% to 90% by mole in a case where the total of all the constitutional units of the carboxy group-containing polymer is set to 100% by mole.

One kind of the constitutional unit having an aromatic ring may be used alone, or two or more kinds thereof may be used in combination.

### <<Constitutional Unit Having Alicyclic Structure>>

It is also preferable that the carboxy group-containing polymer has a constitutional unit having an alicyclic structure in addition to the above-described constitutional unit.

Examples of the alicyclic structure include a tricyclo[5.2.1.0^{2,6}]decane ring structure (also referred to as tetrahydrodicyclopentadiene, where a monovalent group is dicyclopentanyl), a tricyclo[5.2.1.0^{2,6}]decane-3-ene ring structure (also referred to as 5,6-dihydrodicyclopentadiene, where a monovalent group is dicyclopentenyl), an isobornane ring structure (where a monovalent group is isobornyl), an adamantane ring structure (where a monovalent group is adamantyl), and a cyclohexane ring structure (where a monovalent group is cyclohexyl).

Examples of the constitutional unit having an alicyclic structure include a constitutional unit derived from a (meth)acrylate having an alicyclic structure (specifically, dicyclopentanyl (meth)acrylate, dicyclopentenyl (meth)acrylate, isobornyl (meth)acrylate, adamantyl (meth)acrylate, cyclohexyl (meth)acrylate, or the like).

In the carboxy group-containing polymer, the content of the constitutional unit having an alicyclic structure is preferably 0% to 97% by mole, more preferably 0% to 95% by mole, and still more preferably 0% to 90% by mole in a case where the total of all the constitutional units of the carboxy group-containing polymer is set to 100% by mole.

One kind of the constitutional unit having an alicyclic structure may be used alone, or two or more kinds thereof may be used in combination.

### <<Another Constitutional Unit>>

The carboxy group-containing polymer may have another constitutional unit in addition to the above-described constitutional units.

Examples of the other constitutional unit include a constitutional unit derived from methyl (meth)acrylate.

In the carboxy group-containing polymer, the content of the other constitutional unit is preferably 0% to 70% by mole, more preferably 0% to 50% by mole, and still more preferably 0% to 20% by mole in a case where the total of all the constitutional units of the carboxy group-containing polymer is set to 100% by mole.

One kind of the other constitutional unit may be used alone, or two or more kinds thereof may be used in combination.

### (2) Compound B

It is preferable that the composition and the light absorption filter according to the embodiment of the present invention contains together with the above-described compound A as the radical generator, a compound B having a structure that is capable of forming a hydrogen bond with the acid group contained in the compound A (also simply referred to as a “compound B” in the present invention).

The compound B is preferably a compound having such a structure that absorbs ultraviolet rays to be in an excited state, thereby having an increased basicity. In a case where the basicity of the compound B is increased in the excited state, it is possible to form a complex in which the acid group contained in the compound A interacts more strongly with the compound B, and it is possible to increase the efficiency of generating radicals.

A structure contained in the compound B, the structure that is capable of forming a hydrogen bond with the acid group contained in the compound A, may be a whole structure of the compound B or may be a partial structure that constitutes a part of the compound B.

The compound B may be a high-molecular-weight compound (which means a compound having a molecular weight of 5,000 or more) or a low-molecular-weight compound (which means a compound having a molecular weight of less than 5,000), and it is preferably a low-molecular-weight compound.

The molecular weight of the compound B as a low-molecular-weight compound is less than 5,000, and it is preferably less than 1,000, more preferably 500 or less, and still more preferably 350 or less. The lower limit value thereof is not particularly limited; however, it is preferably 65 or more and more preferably 75 or more. Examples of the preferred range of the molecular weight of the compound B which is a low-molecular-weight compound include 65 to 500 and more preferably 75 to 350.

From the viewpoint that the molar absorption coefficient with respect to ultraviolet rays is large, the compound B is preferably an aromatic compound.

Here, the aromatic compound is a compound having one or more aromatic rings.

Only one aromatic ring may be present in the compound B, or a plurality of aromatic rings may be present therein. In a case where a plurality of aromatic rings is present, the aromatic rings may be present, for example, in the side chain or the like of the polymer that constitutes the resin.

The aromatic ring may be any of an aromatic hydrocarbon ring or an aromatic heterocyclic ring. In a case of being an aromatic heterocyclic ring (also referred to as a heteroaromatic ring), it is a compound having one or more (for example, 1 to 4) heteroatoms (at least one among nitrogen atoms, oxygen atoms, sulfur atoms, or the like) as a ring member atom (ring-constituting atom) and preferably has one or more (for example, 1 to 4) nitrogen atoms as a ring member atom.

It is noted that since the unsubstituted aromatic hydrocarbon does not have a structure that is capable of forming a hydrogen bond with the acid group contained in the compound A, the unsubstituted aromatic hydrocarbon does not have a function of generating a radical upon ultraviolet irradiation and does not correspond to the compound B. In addition, since an unsubstituted aromatic hydrocarbon ring in a form in which the unsubstituted aromatic hydrocarbon ring is bonded to the side chain of the polymer that constitutes the resin does not have a structure that is capable of forming a hydrogen bond with the acid group contained in the compound A, the unsubstituted aromatic hydrocarbon ring does not have a function of generating a radical upon ultraviolet irradiation and does not correspond to the compound B.

The number of ring member atoms in the above-described aromatic ring is preferably 5 to 15.

Examples of the above-described aromatic ring include monocyclic aromatic rings such as a pyridine ring, a pyrazine ring, a pyrimidine ring, and a triazine ring; aromatic rings in which two rings are fused, such as a quinoline ring, an isoquinoline ring, a quinoxaline ring, and a quinazoline ring; and aromatic rings in which three rings are fused, such as an acridine ring, a phenanthridine ring, a phenanthroline ring, and a phenazine ring.

The above-described aromatic ring may have one or more (for example, 1 to 5) substituents, and examples of the substituent include an alkyl group, an aryl group, a halogen atom, an acyl group, an alkoxycarbonyl group, an arylcarbonyl group, a carbamoyl group, a hydroxy group, a cyano group, and a nitro group. In addition, in a case where the above-described aromatic ring has two or more substituents, a plurality of substituents may be bonded to each other to form a non-aromatic ring.

In a case where a plurality of aromatic rings (for example, 2 to 5 aromatic rings) forms a series of aromatic ring structures bonded with a structure selected from a single bond, a carbonyl bond, and a multiple bond (for example, a vinylene group which may have a substituent, —C≡C—, —N═N—, and the like), the entire series of aromatic ring structures is regarded as one specific structure.

In addition, it is preferable that one or more of the aromatic rings constituting the series of aromatic ring structures are the above-described heteroaromatic rings.

Specific examples of the compound B include monocyclic aromatic compound such as a pyridine compound (pyridine or a pyridine derivative), a pyrazine compound (pyrazine or a pyrazine derivative), a pyrimidine compound (pyrimidine or a pyrimidine derivative), and a triazine compound (triazine or a triazine derivative); compounds in which two rings are fused to form an aromatic ring, such as a quinoline compound (quinoline or a quinoline derivative), an isoquinoline compound (isoquinoline or an isoquinoline derivative), a quinoxaline compound (quinoxaline or a quinoxaline derivative), and a quinazoline compound (quinazoline or a quinazoline derivative); and compounds in which three or more rings are fused to form an aromatic ring, such as an acridine compound (acridine or an acridine derivative), a phenanthridine compound (phenanthridine or a phenanthridine derivative), a phenanthroline compounds (phenanthroline or a phenanthroline derivative), and a phenazine compounds (phenazine or a phenazine derivative). In the specific examples of the compound B, the compound is used to have a meaning including not only the compound itself but a compound having a substituent (referred to as a “derivative”), including an unsubstituted compound in which a part of the structure has been changed, within a range where the effect of the present invention is not impaired.

It is presumed that this compound B forms a complex with the compound A and generates two molecules of radicals by the following mechanism upon ultraviolet irradiation.
1) The compound B in an excited state is generated by absorbing ultraviolet rays.
2) Positive holes move from the compound B in the excited state to the compound A in the ground state (the electrons of the compound A move to an orbital with lower energy in the two semi occupied molecular orbitals of the compound B in the excited state).
3) The movement of a proton from the compound A to the compound B generates a radical in which a hydrogen radical is added to the compound B and a radical in which a hydrogen radical is eliminated from the compound A.

In a case where the compound A is a compound having a carboxy group, the following reaction further occurs, and a radical is generated by a photodecarboxylation reaction.
4) Carbon dioxide is eliminated from the radical in which the hydrogen radical has been eliminated from the compound A.

Among the above, the compound B is preferably one or more among quinoline compounds (quinoline and a quinoline derivative) and isoquinoline compounds (isoquinoline and an isoquinoline derivative).

The substituent which may be contained in these compounds is preferably an alkyl group, an aryl group, a halogen atom, an acyl group, an alkoxycarbonyl group, an arylcarbonyl group, a carbamoyl group, a hydroxy group, a cyano group, or a nitro group.

In a case where the compound B is a polymer, the compound B may be a polymer in which the above-described specific structure is bonded to a polymer main chain through a single bond or a linking group.

The compound B as a polymer is obtained by, for example, polymerizing a monomer having a heteroaromatic ring (specifically, a (meth)acrylate monomer having a heteroaromatic ring having a vinyl group and/or a specific structure (preferably, a heteroaromatic ring)). As necessary, copolymerization with another monomer may be carried out.

Specific examples of the compound B include quinoline, 2-methylquinoline, 4-methylquinoline, 2,4-dimethylquinoline, 2-methyl-4-phenylquinoline, isoquinoline, 1-methylisoquinoline, 3-methylisoquinoline, and 1-phenylisoquinoline.

From the viewpoint of achieving both the decolorizing properties of the ultraviolet irradiated portion and the durability of the dye in the ultraviolet non-irradiated portion, the content of the compound B is preferably 0.1% to 50% by mass, more preferably 2.0% to 40% by mass, still more preferably 4% to 35% by mass, and particularly preferably 8% to 30% by mass, with respect to the total mass of the composition and the light absorption filter according to the embodiment of the present invention.

In addition, from the viewpoint of achieving both the decolorization properties of the ultraviolet irradiated portion and the durability of the dye in the ultraviolet non-irradiated portion, pKaH (pKa of conjugate acid) which is a scale of the basicity of the compound B is preferably 2.0 to 7.0, more preferably 3.0 to 6.0, and still more preferably 4.3 to 5.5.

In the present invention, the pKa means a negative common logarithm (−log Ka) of the acid dissociation constant (Ka) in a mixed solvent of water/methanol=50/50 (in terms of volume ratio) at 25° C. The pKa can be calculated by dropwise adding a 0.01 mol/L sodium hydroxide aqueous solution to a mixed solution of water/methanol=50/50 (in terms of volume ratio) of a measurement sample (a conjugate acid of the compound B) and reading the amount of the sodium hydroxide aqueous solution that has been dropwise added up to the half-equivalent point.

One kind of the compound B may be used alone, or two or more kinds thereof may be used in combination.

The composition and the light absorption filter according to the embodiment of the present invention may contain a compound that generates a radical upon ultraviolet irradiation (also referred to as a “photoradical generator” in the present invention) in addition to the compound B. The photoradical generator is not particularly limited as long as it is a compound that generates a radical upon ultraviolet irradiation and has a function of decolorizing the above-described dye. It is noted that the radical generated may be a biradical in addition to the typical radical.

As the photoradical generator, a compound commonly used as a photoradical polymerization initiator or a photoradical generator can be used without particular limitation, and examples thereof include an acetophenone generator, a benzoin generator, a benzophenone generator, a phosphine oxide generator, an oxime generator, a ketal generator, an anthraquinone generator, a thioxanthone generator, an azo compound generator, a peroxide generator, a disulfide generator, a lophine dimer generator, an onium salt generator, a borate salt generator, an active ester generator, an active halogen generator, an inorganic complex generator, and a coumarin generator. It is noted that an “XX generator” as the specific example of the photoradical generator may be individually referred to as an “XX compound” or “XX compounds”, and hereinafter, it is referred to as an “XX compound”.

Specific examples, preferred forms, commercially available products, and the like of the photoradical generator are respectively described as the specific examples, preferred forms, commercially available products, and the like of the photoradical initiator in paragraphs [0133] of [0151] JP2009-098658A, and these can be similarly used suitably in the present invention.

The photoradical generator is preferably a compound that generates a radical upon intramolecular cleavage or a compound that abstracts a hydrogen atom from a compound present in the vicinity thereof to generate a radical, and it is more preferably a compound that abstracts a hydrogen atom from a compound present in the vicinity thereof to generate a radical, from the viewpoint of further improving the decolorization rate.

The above-described compound that generates a radical upon intramolecular cleavage (hereinafter, also referred to as an “intramolecular cleavage type photoradical generator”) means a compound that generates a radical, where the compound absorbing light undergoes bonding cleavage in a homolytic manner.

Examples of the intramolecular cleavage type photoradical generator include an acetophenone compound, a benzoin compound, a phosphine oxide compound, an oxime compound, a ketal compound, an azo compound, a peroxide compound, a disulfide compound, an onium salt compound, a borate salt compound, an active ester compounds, an active halogen compound, an inorganic complex compound, and a coumarin compound. Among these, an acetophenone compound, a benzoin compound, or a phosphine oxide compound, which is a carbonyl compound, is preferable. The Norrish type I reaction is known as a photodecomposition reaction of an intramolecular cleavage type carbonyl compound, and this reaction can be referenced as a radical generation mechanism.

The above-described compound that abstracts a hydrogen atom from a compound present in the vicinity thereof to generate a radical (hereinafter, also referred to as a “hydrogen abstraction type photoradical generator”) means a carbonyl compound in an excited triplet state obtained upon light absorption that abstracts a hydrogen atom from a compound present in the vicinity thereof to generate a radical.

A carbonyl compound is known as the hydrogen abstraction type photoradical generator, and examples thereof include a benzophenone compound, an anthraquinone compound, and a thioxanthone compound. The Norrish type II reaction is known as a photodecomposition reaction of a hydrogen abstraction type carbonyl compound, and this reaction can be referenced as a radical generation mechanism.

Examples of the compound present in the vicinity thereof include various components present in the composition and the light absorption filter according to the embodiment of the present invention, such as a resin, a dye, and a radical generator.

The compound present in the vicinity which is a compound having a radical by a hydrogen atom being abstracted therefrom. Since a dye from which a hydrogen atom has been abstracted by the hydrogen abstraction type photoradical generator which is an active compound having a radical, the dye may be faded or decolorized through a reaction such as the decomposition of the dye having the radical.

In addition, in a case where the hydrogen abstraction type photoradical generator abstracts a hydrogen atom in the molecule, a biradical is generated.

The hydrogen abstraction type photoradical generator is preferably a benzophenone compound from the viewpoint of the quantum yield of the hydrogen abstraction reaction.

Various examples of the photoradical generator are also described in “Latest UV Curing Technology”, TECHNICAL INFORMATION INSTITUTE CO. LTD., 1991, p. 159, and “Ultraviolet Curing System”, written by Kiyomi Kato, 1989, published by SOGO GIJUTSU CENTER, p. 65 to 148, which can be also suitably used in the present invention.

In the photoradical generator, the maximal absorption wavelength of the ultraviolet ray to be absorbed is preferably in a range of 250 to 400 nm, more preferably in a range of 240 to 400 nm, and still more preferably in a range of 270 to 400 nm.

In a case where the photoradical generator is a benzophenone compound, the wavelength of the absorption maximum attributed to the n-π* transition, which is located on the longest wavelength side, is preferably in a range of 260 to 400 nm and more preferably in a range of 285 to 345 nm. The wavelength of the absorption maximum attributed to π-π*, which is located on the second longest wavelength side, is preferably in a range of 240 to 380 nm and more preferably in a range of 270 to 330 nm. In a case where the absorption maximum wavelength is set in the above range, the light of a light source used at the time of exposure, such as a metal halide lamp, is absorbed well. On the other hand, in a case of being incorporated into a display device, it is difficult to absorb an ultraviolet ray incident from the outside, and thus it is possible to achieve both the light resistance of the unexposed portion and the decolorizing properties of the exposed portion.

Among the benzophenone compounds, examples of the photoradical generator having absorption in a longer wavelength range include an alkoxybenzophenone compound.

In general, the maximal absorption wavelength of the ultraviolet ray absorbed by the photoradical generator is preferably separated by 30 nm or more from the main absorption wavelength band of the dye that has a main absorption wavelength band in a wavelength range of 400 to 700 nm. The upper limit value thereof is not particularly limited.

Examples of the commercially available photocleavage type photoradical generator include “Irgacure 651”, “Irgacure 184”, “Irgacure 819”, “Irgacure 907”, “Irgacure 1870” (a mixed initiator of CGI-403/Irgacure 184=7/3), “Irgacure 500”, “Irgacure 369”, “Irgacure 1173”, “Irgacure 2959”, “Irgacure 4265”, “Irgacure 4263”, “Irgacure 127”, or “OXE01”, all of which are product names, manufactured by BASF SE (formerly Ciba Specialty Chemicals Inc.); additionally, “Kayacure DETX-S”, “Kayacure BP-100”, “Kayacure BDMK”, “Kayacure CTX”, “Kayacure BMS”, “Kayacure 2-EAQ”, “Kayacure ABQ”, “Kayacure CPTX”, “Kayacure EPD”, “Kayacure ITX”, “Kayacure QTX”, “Kayacure BTC”, and “Kayacure MCA”, manufactured by Nippon Kayaku Co., Ltd.; and more additionally “Esacure (KIP100F, KB1, EB3, BP, X33, KT046, KT37, KIP150, and TZT)” manufactured by Sartomer Company Inc. In addition, preferred examples thereof include a combination of two or more of these.

In a case where the composition and the light absorption filter according to the embodiment of the present invention contain a photoradical generator, the content of the photoradical generator in the composition and the light absorption filter according to the embodiment of the present invention is preferably 0.01% to 30% by mass and more preferably 0.1% to 20% by mass.

From the viewpoint of further improving the decolorization rate, the blending amount of the radical generator in the composition and the light absorption filter according to the embodiment of the present invention is preferably 0.1 to 20 mol with respect to 1 mol of the dye. The lower limit value thereof is more preferably 0.25 mole or more and still more preferably 0.50 mole or more. The upper limit value thereof is more preferably 17.5 mol or less and still more preferably 15 mol or less. The blending amount of the radical generator referred to herein means the blending amount of the photoradical generator or the blending amount of the compound B described above, and does not include the blending amount of the compound A.

The composition and the light absorption filter according to the embodiment of the present invention may contain one kind of radical generator or two or more kinds thereof.

### <Resin>

The resin contained in the composition and the light absorption filter according to the embodiment of the present invention (hereinafter, also referred to as a “matrix resin”) is not limited as long as it can disperse (preferably dissolve) the above-described dye, can exhibit the decolorization action of the dye due to the radical generated from a compound (preferably, the compound B that has been hydrogen-bonded to the acid group contained in the compound A) that generates a radical upon ultraviolet irradiation, and has desired light transmittance (the light transmittance is preferably 80% or more in the visible range in a wavelength range of 400 to 800 nm).

As a polymer that constitutes the above-described resin, various polymers can be used. However, from the viewpoint that the molecular weight of the resin is not easily reduced upon ultraviolet irradiation, a polymer having an aromatic ring or an alicyclic structure in the side chain is preferable, and a (meth)acrylic polymer containing a constitutional unit having an aromatic ring or an alicyclic structure is more preferable. Among the above, from the viewpoint of further improving the decolorization rate and further improving the heat resistance and the light resistance as well, a (meth)acrylic polymer containing a constitutional unit having an alicyclic structure in the side chain is still more preferable.

Here, the (meth)acrylic polymer refers to a polymer containing at least one of a constitutional unit derived from (meth)acrylic acid or a constitutional unit derived from (meth)acrylic acid ester. It is noted that the constitutional unit derived from (meth)acrylic acid is a constitutional unit having a carboxy group as the acid group in the compound A, in a case where the polymer contains a constitutional unit derived from (meth)acrylic acid, and the polymer corresponds to a polymer in which the compound A is chemically bonded to the above-described polymer that constitutes the resin.

In addition, in the present disclosure, the “main chain” represents a relatively longest bonding chain in a molecule of a high-molecular-weight compound, and the “side chain” represents an atomic group branched from the main chain.

Examples of the monomer from which a constitutional unit having an aromatic ring is derived include benzyl acrylate, benzyl methacrylate, naphthyl acrylate, naphthyl methacrylate, naphthyl methyl acrylate, and naphthyl methyl methacrylate. In a case where the polymer does not contain a constitutional unit derived from (meth)acrylic acid, the content of the constitutional unit having an aromatic ring is preferably 5% to 100% by mass, more preferably 10% to 100% by mass, and still more preferably 20% to 100% by mass, with respect to the total mass of the polymer.

Examples of the monomer from which a constitutional unit having an alicyclic structure is derived include dicyclopentanyl (meth)acrylate, cyclohexyl (meth)acrylate, and isobornyl (meth)acrylate.

In a case where the polymer contains a constitutional unit having an alicyclic structure, the content of the constitutional unit having an alicyclic structure is preferably 1% to 90% by mass, more preferably 5% to 90% by mass, and still more preferably 5% to 80% by mass, with respect to the total mass of the polymer.

In addition, in the composition and the light absorption filter according to the embodiment of the present invention, the polymer that constitutes the resin may contain a constitutional unit bonded to the compound A having an acid group. The constitutional unit bonded to the compound A having an acid group is preferably a constitutional unit derived from (meth)acrylic acid. The content of the constitutional unit derived from (meth)acrylic acid is preferably 1% to 70% by mass and more preferably 1% to 60% by mass with respect to the total mass of the polymer. More preferably, the description of the content of the constitutional unit having the carboxy group of the carboxy group-containing polymer as the compound A is applied.

In a case where the polymer that constitutes the resin contains a constitutional unit bonded to the compound A having an acid group, the descriptions regarding the content of the constitutional unit having the carboxy group of the carboxy group-containing polymer as the compound A and the content of the constitutional unit having an aromatic ring and the content of the constitutional unit having an alicyclic structure are applied to the content of the constitutional unit bonded to the compound A having an acid group, the content of the constitutional unit having an aromatic ring, and the content of the constitutional unit having an alicyclic structure.

From the viewpoint of adjusting the glass transition temperature and the like, the polymer that constitutes the resin may contain a constitutional unit that has an alkyl group having 1 to 14 carbon atoms. Examples of the constitutional unit having an alkyl group having 1 to 14 carbon atoms include a constitutional unit derived from an alkyl (meth)acrylate, such as methyl (meth)acrylate, ethyl (meth)acrylate, n-propyl (meth)acrylate, isopropyl (meth)acrylate, n-butyl (meth)acrylate, t-butyl (meth)acrylate, sec-butyl (meth)acrylate, pentyl (meth)acrylate, 2-ethylhexyl (meth)acrylate, 2-ethylbutyl (meth)acrylate, n-octyl (meth)acrylate, isooctyl (meth)acrylate, isononyl (meth)acrylate, lauryl (meth)acrylate, or tetradecyl (meth)acrylate. In the present invention, one kind of the constitutional unit having an alkyl group having 1 to 14 carbon atoms may be used alone, or two or more kinds thereof may be used in combination. The content of the constitutional unit having an alkyl group having 1 to 14 carbon atoms is preferably such that an amount of 0% by mass to 95% by mass is contained with respect to the total mass of the polymer that constitutes the resin.

The weight-average molecular weight (Mw) of the polymer that constitutes the resin is preferably 10,000 or more, more preferably 10,000 to 200,000, and still more preferably 15,000 to 150,000.

### <Other Components>

The composition and the light absorption filter according to the embodiment of the present invention may contain an antifading agent, a matting agent, a leveling agent (surfactant), and the like in addition to the dye including at least the azo-based coloring agent represented by General Formula (i) described above, the compound that generates a radical upon ultraviolet irradiation described above, and the above-described resin (matrix polymer).

### <Antifading Agent>

In the composition and the light absorption filter according to the embodiment of the present invention, it is preferable that the antifading agent does not inhibit the decolorization due to ultraviolet irradiation but has an effect of suppressing the dye decomposition due to visible light.

The compound represented by General Formula (IV) below can be preferably used as the antifading agent.

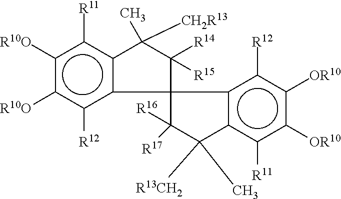

In Formula (IV), R¹⁰ represents an alkyl group, an alkenyl group, an aryl group, a heterocyclic group, or a group represented by R¹⁸CO—, R¹⁹SO₂—, or R²⁰NHCO—. Here, R¹⁸, R¹⁹, and R²⁰ each independently represent an alkyl group, an alkenyl group, an aryl group, or a heterocyclic group. R¹¹ and R¹² each independently represent a hydrogen atom, a halogen atom, an alkyl group, an alkenyl group, an alkoxy group, or an alkenyloxy group, and R¹³, R¹⁴, R¹⁵, R¹⁶, and R¹⁷ each independently represent a hydrogen atom, an alkyl group, an alkenyl group, or an aryl group.

However, the alkyl group in R¹⁰ to R²⁰ includes an aralkyl group.

The compound represented by General Formula (IV) is the same as the compound represented by General Formula (IV) described in paragraphs [0215] to [0221] of WO2021/221122A. Therefore, for the description of each substituent in General Formula (IV) and the specific examples of the compound represented by General Formula (IV), the description of paragraphs [0217] to [0221] of WO2021/221122A can be applied as it is.

As the antifading agent, the compound represented by General Formula [III] below can also be preferably used.

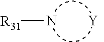

In General Formula [III], R₃₁ represents an aliphatic group or an aromatic group, and Y represents a group of non-metal atoms necessary for forming a 5- to 7-membered ring with a nitrogen atom.

The compound represented by General Formula [III] is the same as the compound represented by General Formula [III] described in paragraphs [0223] to [0227] of WO2021/221122A. Therefore, for the description of each substituent in General Formula [III] and the specific examples of the compound represented by General Formula [III], the description of paragraphs [0225] to [0227] of WO2021/221122A can be applied as it is.

Further, in addition to the specific examples, specific examples of the compound represented by General Formula [III] above can include exemplary compounds B-1 to B-65 described on pages 8 to 11 of JP1990-167543A (JP-H2-167543A), and exemplary compounds (1) to (120) described on pages 4 to 7 of JP1988-95439A (JP-S63-95439A).

The content of the antifading agent in the composition and the light absorption filter according to the embodiment of the present invention is preferably 1% to 15% by mass, more preferably 5% to 15% by mass, still more preferably 5% to 12.5% by mass, and particularly preferably 10% to 12.5% by mass.

In a case where the antifading agent is contained within the above-described preferred range, it is possible to improve the light resistance of the dye (the coloring agent) without causing side effects such as discoloration.

### (Matting Agent)

In order to impart sliding properties and prevent blocking, fine particles may be added to the surface of the light absorption filter according to the embodiment of the present invention as long as the effect of the present invention is not impaired. As the fine particles, silica (silicon dioxide, SiO₂) of which the surface is coated with a hydrophobic group and which has an aspect of secondary particles is preferably used. As the fine particles, in addition to or instead of silica, fine particles of titanium dioxide, aluminum oxide, zirconium oxide, calcium carbonate, talc, clay, calcined kaolin, calcined calcium silicate, hydrated calcium silicate, aluminum silicate, magnesium silicate, and calcium phosphate may be used. Examples of the commercially available product of the fine particles include R972 or NX90S (product name, both manufactured by Nippon Aerosil Co., Ltd.).

The fine particles function as a so-called matting agent, and the addition of the fine particles forms minute unevenness on the surface of the light absorption filter according to the embodiment of the present invention. Due to the unevenness, even in a case where the light absorption filters according to the embodiment of the present invention overlap with each other or the light absorption filter according to the embodiment of the present invention and other films overlap with each other, the films do not stick to each other and sliding properties are secured.

In a case where the light absorption filter according to the embodiment of the present invention contains a matting agent as fine particles, the effect of improving sliding properties and blocking properties is particularly large in the fine unevenness due to the protrusions in which fine particles protrude from the filter surface in a case where there are 10⁴/mm² or more of protrusions having a height of 30 nm or more.

It is preferable to apply the matting agent (fine particles) particularly onto the surface layer in order to improve the blocking properties and the sliding properties. Examples of the method of applying fine particles onto the surface layer include methods such as multilayer casting and coating.

The content of the matting agent in the composition and the light absorption filter according to the embodiment of the present invention is appropriately adjusted depending on the purpose.

However, in a case where a gas barrier layer described later is provided in the light absorption filter according to the embodiment of the present invention, the above-described matting agent fine particles are preferably applied onto the surface of the light absorption filter in contact with the gas barrier layer as long as the effect of the present invention is not impaired.

### (Leveling Agent)

A leveling agent (surfactant) can be appropriately mixed into the composition and the light absorption filter according to the embodiment of the present invention. As the leveling agent, a commonly used compound can be used, and a fluorine-containing surfactant is particularly preferable. Specific examples thereof include the compounds described in paragraphs [0028] to [0056] of JP2001-330725A. In addition, as the commercially available product, MEGAFACE F (product name) series manufactured by DIC Corporation can also be used.

The content of the leveling agent in the composition and the light absorption filter according to the embodiment of the present invention is appropriately adjusted depending on the purpose.

The composition and the light absorption filter according to the embodiment of the present invention may contain, in addition to each component described above, a low-molecular plasticizer, an oligomer-based plasticizer, a retardation modifier, a deterioration preventing agent, a peeling accelerating agent, an infrared absorbing agent, an antioxidant, a filler, and a compatibilizer.

In addition, the composition and the light absorption filter according to the embodiment of the present invention may contain the reaction accelerating agent or the reaction retarder described in paragraphs [0020] and [0021] of JP1997-286979A (JP-H09-286979A).

### <Manufacturing Method for Light Absorption Filter>

The light absorption filter according to the embodiment of the present invention can be produced by a solution film-forming method, a melt extrusion method, or a method of forming a coating layer on a base material film (support film) (coating method) according to any method, according to a conventional method, and stretching can also be appropriately combined. The light absorption filter according to the embodiment of the present invention is preferably produced by a coating method.

For the solution film-forming method and melt extrusion method described above, the descriptions regarding the solution film-forming method and the melt extrusion method in [0197] of WO2021/132674A can be applied as they are.

### (Coating Method)

In the coating method, a solution of a material of the light absorption filter is applied to a support film to form a coating layer. A release agent or the like may be appropriately applied to the surface of the support film in advance in order to control the adhesiveness to the coating layer. The coating layer can be used by peeling off the support film after being laminated with another member while interposing an adhesive layer in a later step. Any adhesive can be appropriately used as the adhesive constituting the adhesive layer. The whole support film can be appropriately stretched in a state where a solution of the material of the light absorption filter is applied on the support film or in a state where a coating layer is laminated on the support film.

A solvent that is used for the solution of the material of the light absorption filter can be appropriately selected from the viewpoints that the material of the light absorption filter can be dissolved or dispersed, that a uniform surface shape can be easily achieved during the coating step and drying step, liquid storability can be secured, and that a proper saturated vapor pressure is provided.

### —Addition of Dye (Coloring Agent) and Radical Generator—

The timing of adding the dye including at least the azo-based coloring agent represented by General Formula (i) and the above-described radical generator to the material of the light absorption filter is not particularly limited as long as the addition is made at the time of film formation. For example, the dye may be added at the time of synthesizing the matrix polymer or may be mixed with the material of the light absorption filter at the time of preparing the coating liquid for the material of the light absorption filter. It is noted that in a case where the radical generator includes a combination of the compound A and the compound B, and the compound A is bonded to the polymer that constitutes the resin, the compound A is added at the time of adding the polymer that constitutes the resin.

### —Support Film—

The support film that is used for forming the light absorption filter according to the embodiment of the present invention by a coating method or the like preferably has a film thickness of 5 to 100 μm, more preferably 10 to 75 μm, and still more preferably 15 to 55 μm. In a case where the film thickness is equal to or larger than the above-described preferred lower limit value, sufficient mechanical strength can be easily secured, and failures such as curling, wrinkling, and buckling are less likely to occur. In addition, in a case where the film thickness is equal to or smaller than the above-described preferred upper limit value, in the storage of a multi-layer film of the support film and the light absorption filter according to the embodiment of the present invention, for example, in the form of a long roll, the surface pressure applied to the multi-layer film is easily adjusted to be in an appropriate range, and adhesion defect is less likely to occur.

The surface energy of the support film is not particularly limited, and by adjusting the relationship between the surface energy of the material of the light absorption filter according to the embodiment of the present invention or the coating solution and the surface energy of the surface of the support film on which the light absorption filter according to the embodiment of the present invention is to be formed, the adhesive force between the light absorption filter according to the embodiment of the present invention and the support film can be adjusted. In a case where the surface energy difference is reduced, the adhesive force tends to increase, and in a case where the surface energy difference is increased, the adhesive force tends to decrease, and thus the surface energy can be set appropriately.

The surface unevenness of the support film is not particularly limited, and depending on the relationship between the surface energy of the light absorption filter according to the embodiment of the present invention surface, the hardness, and the surface unevenness, and the surface energy and hardness of the surface of the support film opposite to the side on which the light absorption filter according to the embodiment of the present invention is formed, for example, in order to prevent adhesion defect in a case where the multi-layer film of the support film and the light absorption filter according to the embodiment of the present invention is stored in the form of a long roll, the surface unevenness of the support film can be adjusted. In a case where the surface unevenness is increased, adhesion defect tends to be suppressed, and in a case where the surface unevenness is reduced, the surface unevenness of the light absorption filter according to the embodiment of the present invention tends to be decreased and the haze of the light absorption filter according to the embodiment of the present invention tends to be small. Thus, the surface unevenness can be set appropriately.

For such a support film, any material and film can be appropriately used. Specific examples of the material include a polyester-based polymer (including a polyethylene terephthalate-based polymer), an olefin-based polymer, a cycloolefin-based polymer, a (meth)acrylic polymer, a cellulose-based polymer, and a polyamide-based polymer. In addition, a surface treatment can be appropriately carried out for the intended purpose of adjusting the surface properties of the support film. For example, a corona treatment, a room temperature plasma treatment, or a saponification treatment can be carried out to decrease the surface energy, and a silicone treatment, a fluorine treatment, an olefin treatment, or the like can be carried out to raise the surface energy.

### Film Thickness of Light Absorption Filter According to Embodiment of Present Invention

The film thickness of the light absorption filter according to the embodiment of the present invention is not particularly limited, and it is preferably 1 to 18 μm, more preferably 1 to 12 μm, and still more preferably 2 to 8 μm. In a case where the film thickness is equal to or smaller than the above-described preferred upper limit value, the decrease in the degree of polarization due to the fluorescence emitted by a dye (a coloring agent) can be suppressed by adding the dye to the thin film at a high concentration. In addition, the effect of the quencher is likely to be exhibited. On the other hand, in a case where the film thickness is equal to or larger than the above-described preferred lower limit value, it is easy to maintain the evenness of the in-plane absorbance.

In the present invention, the film thickness of 1 to 18 μm means that the thickness of the light absorption filter according to the embodiment of the present invention is within a range of 1 to 18 μm in a case of being measured at any portion. The same applies to the film thicknesses of 1 to 12 μm and 2 to 8 μm. The film thickness can be measured with an electronic micrometer manufactured by Anritsu Corporation.

### Absorbance of Light Absorption Filter of According to Embodiment of Present Invention

In the light absorption filter according to the embodiment of the present invention, the absorbance at the maximal absorption wavelength at which the highest absorbance is exhibited at a wavelength of 400 to 700 nm (hereinafter, also simply referred to as “Ab(λₘₐₓ)”) is preferably 0.3 or more, more preferably 0.5 or more, and still more preferably 0.7 or more.

However, the absorbance of the light absorption filter according to the embodiment of the present invention can be adjusted by the type, adding amount, or film thickness of the dye.

In the light absorption filter according to the embodiment of the present invention, a decolorization rate upon ultraviolet irradiation at 25° C to 37° C is preferably 85% or more, more preferably 87% or more, and still more preferably 90% or more. The upper limit value thereof is not particularly limited, and it is preferably 100%.

The decolorization rate is calculated according to the following expression using the values of Ab(λₘₐₓ) before and after the ultraviolet irradiation test.

Decolorization rate (%)=100−(Ab(λₘₐₓ) after ultraviolet irradiation/Ab(λₘₐₓ) before ultraviolet irradiation)×100%

Here, in the ultraviolet irradiation test, an ultra-high pressure mercury lamp (for example, manufactured by HOYA Corporation, product name: UL750) is used under atmospheric pressure (101.33 kPa) to irradiate the light absorption filter with ultraviolet rays at an illuminance of 100 mW/cm² and an irradiation amount of 750 to 3000 mJ/cm² at near room temperature (10° C to 37° C).

The absorbance, the ultraviolet irradiation test, and the decolorization rate can be measured and calculated according to the methods described in Examples.

In addition, it is preferable that the light absorption filter according to the embodiment of the present invention hardly causes absorption (secondary absorption) derived from a new coloration structure associated with the decomposition of the coloring agent.

For example, the presence or absence of the absorption derived from the new coloration structure associated with the decomposition of the coloring agent can be checked based on the ratio of the absorbance at a specific wavelength to the above Ab(λₘₐₓ). As the specific wavelength, a wavelength at which the coloring agent before ultraviolet irradiation seldom exhibits absorption but new absorption due to the decomposition of the coloring agent is observed is selected.

As a specific example, as described in Examples described later, the presence or absence of the absorption derived from the new coloration structure associated with the decomposition of the coloring agent can be checked based on the ratio of the absorbance at a specific wavelength to the above Ab(λₘₐₓ). As the specific wavelength, a wavelength at which the coloring agent before ultraviolet irradiation seldom exhibits absorption but new absorption due to the decomposition of the coloring agent is observed is selected. As a specific example, as described in Examples described later, the presence or absence of the absorption derived from a new coloration structure associated with the decomposition of the coloring agent can be checked based on the ratio of the absorbance at a wavelength of 450 nm to the above Ab(λₘₐₓ) (hereinafter, also simply referred to as “Ab(450)”). That is, it is meant that the smaller the value obtained by subtracting the ratio of the following (I) from the ratio of the following (II), the less frequently the absorption derived from the new coloration structure associated with the decomposition of the coloring agent occurs. This value is preferably less than 8.5%, more preferably 7.0% or less, still more preferably 5.0% or less, particularly preferably 3.0% or less, among which 1.0% or less is preferable. The lower limit value thereof is not particularly limited; however, it is practically −10% or more and preferably −6% or more from the viewpoint of making valid the evaluation related to the presence or absence of the secondary absorption associated with the decomposition of the coloring agent.

{Ab(450) before ultraviolet irradiation/Ab(λₘₐₓ) before ultraviolet irradiation}×100% (I)

{Ab(450) after ultraviolet irradiation/Ab(λₘₐₓ) before ultraviolet irradiation}×100% (II)

The checking of the presence or absence of the absorption derived from the new coloration structure associated with the decomposition of the coloring agent can be carried out by the measurement and the calculation according to the method described in Examples.

Further, as described in Examples described later, the value of the absorbance at a wavelength of 650 nm (hereinafter, also simply referred to as “Ab(650)”) is used instead of the absorbance at a wavelength of 450 nm to obtain a value obtained by subtracting the ratio of the following (III) from the ratio of the following (IV), whereby the evaluation can be carried out. The preferred range of the value obtained by subtracting the ratio of the following (III) from the ratio of the following (IV) has the same meaning as the value obtained by subtracting the ratio of the above expression (I) from the ratio of the above expression (II).

{Ab(650) before ultraviolet irradiation/Ab(λₘₐₓ) before ultraviolet irradiation}×100% (III)

{Ab(650) after ultraviolet irradiation/Ab(λₘₐₓ) before ultraviolet irradiation}×100% (IV)

Here, the description of the ultraviolet irradiation test regarding the above decolorization rate can be preferably applied to the ultraviolet irradiation test.

The checking of the presence or absence of the absorption derived from the new coloration structure associated with the decomposition of the coloring agent can be carried out by the measurement and the calculation according to the method described in Examples.

The light absorption filter according to the embodiment of the present invention can exhibit excellent decolorizing properties in a case where both the above-described decolorization rate and the above-described value for checking the presence or absence of the absorption derived from the new coloration structure associated with the decomposition of the coloring agent satisfy a preferred range.

The light absorptive portion having a light absorption effect in the optical filter according to the embodiment of the present invention preferably satisfies the above description of Ab(λₘₐₓ) related to the light absorption filter according to the embodiment of the present invention.

### Treatment of Light Absorption Filter According to Embodiment of Present Invention

The light absorption filter according to the embodiment of the present invention may be subjected to a hydrophilic treatment by any of glow discharge treatment, corona discharge treatment, or alkali saponification treatment, and a corona discharge treatment is preferably used. It is also preferable to apply the method disclosed in JP1994-94915A (JP-H6-94915A) and JP1994-118232A (JP-H6-118232A).

As necessary, the obtained film may be subjected to a heat treatment step, a superheated steam contact step, an organic solvent contact step, or the like. In addition, a surface treatment may be appropriately carried out.

In addition, as a pressure-sensitive adhesive layer, a layer consisting of a pressure-sensitive adhesive composition in which a (meth)acrylic resin, a styrene-based resin, a silicone-based resin, or the like is used as a base polymer, and a crosslinking agent such as an isocyanate compound, an epoxy compound, or an aziridine compound is added thereto can be applied.

Preferably, the description regarding the pressure-sensitive adhesive layer in the OLED display device described later can be applied.

### <Gas Barrier Layer>

The light absorption filter according to the embodiment of the present invention may have a gas barrier layer on at least one surface. In a case where the light absorption filter according to the embodiment of the present invention has a gas barrier layer, the light absorption filter according to the embodiment of the present invention can be a light absorption filter that achieves both excellent light resistance and excellent heat resistance exhibited by the light absorption property-eliminated portion obtained by ultraviolet irradiation, and can be suitably used for producing an optical filter described later.

The material that forms the gas barrier layer is not particularly limited, and examples thereof include an organic material (preferably a crystalline resin) such as polyvinyl alcohol or polyvinylidene chloride, an organic-inorganic hybrid material such as a sol-gel material, and an inorganic material such as SiO₂, SiOₓ, SiON, SiNₓ, or Al₂O₃. The gas barrier layer may be a single layer or a multilayer. In the case of a multilayer, examples thereof include configurations such as an inorganic dielectric multilayer film and a multilayer film obtained by alternately laminating organic-based materials and inorganic-based materials.

In a case where the light absorption filter according to the embodiment of the present invention includes the gas barrier layer at least on a surface that comes into contact with air in a case where the light absorption filter according to the embodiment of the present invention is used, it is possible to suppress a decrease in the absorption intensity of the dye in the light absorption filter according to the embodiment of the present invention. As long as the gas barrier layer is provided at an interface of the light absorption filter according to the embodiment of the present invention in contact with air, the gas barrier layer may be provided on only one surface of the light absorption filter according to the embodiment of the present invention, or may be provided on both surfaces.

Among the above, in a case of a configuration in which the gas barrier layer contains a crystalline resin, the gas barrier layer contains a crystalline resin, and it is preferable that the thickness of the layer is 0.1 μm to 10 μm and the oxygen permeability of the layer is 60 cc/m²·day·atm or less.

In the gas barrier layer, the “crystalline resin” is a resin having a melting point that undergoes a phase transition from a crystal to a liquid in a case where the temperature is raised, and it can impart gas barrier properties related to oxygen gas to the gas barrier layer.

“The gas barrier layer containing a crystalline resin, where the thickness of the layer is 0.1 μm to 10 μm and the oxygen permeability of the layer is 60 cc/m²·day·atm or less” described above is the same as the gas barrier layers described in [0180] to [0184] of WO2022/149510A, and the descriptions regarding the gas barrier layer which is described in [0180] of WO2022/149510A, can be applied as they are.

### <Manufacturing Method for Gas Barrier Layer>

The method of forming the gas barrier layer is not particularly limited, and examples thereof include a producing method according to a conventional method according to a casting method such as spin coating or slit coating, for example, in a case of an organic material. In addition, examples thereof can include a method of bonding a commercially available resin gas barrier film or a resin gas barrier film produced in advance to the light absorption filter according to the embodiment of the present invention. In addition, examples thereof include a plasma enhanced chemical vapor deposition (PECVD) method, a sputtering method and a vapor deposition method in a case of an inorganic material.

In a case where the above-described gas barrier layer is provided in the light absorption filter according to the embodiment of the present invention, for example, a method of directly producing the above-described gas barrier layer on the light absorption filter according to the embodiment of the present invention produced according to the above-described production method is included. In this case, it is also preferable to apply a corona treatment to the surface of the light absorption filter according to the embodiment of the present invention to which the gas barrier layer is provided.

In addition, in a case where the above-described optional optical functional film is provided, it is also preferable to carry out bonding while interposing a pressure-sensitive adhesive layer. For example, it is also preferable that a gas barrier layer is provided on the light absorption filter according to the embodiment of the present invention and then bonded to an optical functional film while interposing a pressure-sensitive adhesive layer.

### <Optical Functional Film>

The light absorption filter according to the embodiment of the present invention may appropriately have the gas barrier layer or any optical functional film as long as the effect of the present invention is not impaired.

The optional optical functional film is not particularly limited in terms of any of the optical properties and the materials, and a film containing (or containing as a main component) at least any of a cellulose ester resin, an acrylic resin, a cyclic olefin resin, or a polyethylene terephthalate resin can be preferably used. It is noted that an optically isotropic film or an optically anisotropic phase difference film may be used.

For the above optional optical functional films, for example, Fujitac TD80UL (product name, manufactured by FUJIFILM Corporation) or the like can be used as a film containing a cellulose ester resin.

Regarding the optional optical functional film, as those containing an acrylic resin, an optical film containing a (meth)acrylic resin containing a styrene-based resin described in JP4570042B, an optical film containing a (meth)acrylic resin having a glutarimide ring structure in a main chain described in JP5041532B, an optical film containing a (meth)acrylic resin having a lactone ring structure described in JP2009-122664A, and an optically functional film containing a (meth)acrylic resin having a glutaric anhydride unit described in JP2009-139754A can be used.

In addition, regarding the optional optical functional films, as those containing a cyclic olefin resin, cyclic olefin-based resin film described in paragraphs [0029] and subsequent paragraphs of JP2009-237376A, and cyclic olefin resin film containing an additive reducing Rth described in JP4881827B, and JP2008-063536A can be used.

### [Optical Filter]

The optical filter according to the embodiment of the present invention is obtained by subjecting the light absorption filter according to the embodiment of the present invention to mask exposure by ultraviolet irradiation.

The optical filter according to the embodiment of the present invention has a light absorptive portion having a light absorption effect and a site where the light absorption property disappears (light absorption property-eliminated portion) exhibiting excellent heat resistance, according to a mask exposure pattern (hereinafter, also referred to as a “mask pattern”).

That is, in a case where the light absorption filter according to the embodiment of the present invention is subjected to mask exposure by ultraviolet irradiation, the masked portion of the light absorption filter according to the embodiment of the present invention is not exposed and present as a light absorptive portion having a light absorption effect, whereas the unmasked portion is exposed and is a light absorption property-eliminated portion.

The light absorptive portion can exhibit a desired absorbance.

In addition, since the azo-based coloring agent represented by General Formula (i) contained in the light absorption filter according to the embodiment of the present invention exhibits an excellent decolorization rate and almost no secondary absorption occurs due to decomposition of the dye, the light absorption property-eliminated portion can exhibit optical properties close to colorless and can exhibit light transmittance close to colorless even in a case of being exposed to heat, since the light absorption property-eliminated portion exhibits excellent heat resistance.

### <Manufacturing Method for Optical Filter>

The optical filter according to the embodiment of the present invention can be obtained by irradiating the light absorption filter according to the embodiment of the present invention with an ultraviolet ray to carry out mask exposure.

The mask pattern can be appropriately adjusted so that the optical filter according to the embodiment of the present invention having a desired pattern consisting of a light absorptive portion and a light absorption property-eliminated portion can be obtained.

The conditions of ultraviolet irradiation can be appropriately adjusted so that the optical filter according to the embodiment of the present invention having a light absorption property-eliminated portion can be obtained. For example, the pressure condition can be performed under atmospheric pressure (101.33 kPa), the temperature condition can be performed under mild temperature conditions without heating at near room temperature (10° C to 37° C) or the like or by heating at a low temperature, the lamp output can be set to 10 to 320 W/cm, and as the lamp to be used, a metal halide lamp, a mercury lamp such as an ultra-high pressure mercury lamp, or the like can be used. In addition, the irradiation amount can be set to 200 to 3,500 mJ/cm².

The optical filter according to the embodiment of the present invention may have an optical functional film described in the light absorption filter according to the embodiment of the present invention.

In addition, the optical filter according to the embodiment of the present invention may have a layer containing an ultraviolet absorbing agent. As the ultraviolet absorbing agent, a commonly used compound can be used without particular limitation, and examples thereof include an ultraviolet absorbing agent in the ultraviolet absorbing layer described later.

The resin constituting the layer containing the ultraviolet absorbing agent is also not particularly limited, and examples thereof include a resin in the ultraviolet absorbing layer described later.

The content of the ultraviolet absorbing agent in the layer containing the ultraviolet absorbing agent is appropriately adjusted according to the intended purpose.

### [OLED Display Device]

The organic electroluminescent display device according to the embodiment of the present invention (referred to as an organic electroluminescence (EL) display device or an organic light emitting diode (OLED) display device, and abbreviated as an OLED display device in the present invention) includes the optical filter according to the embodiment of the present invention.

As another configuration of the OLED display device according to the embodiment of the present invention, the configuration of the generally used OLED display device can be used without particular limitation, as long as the optical filter according to the embodiment of the present invention is included. The configuration example of the OLED display device according to the embodiment of the present invention is not particularly limited, and examples thereof include a display device including glass, a layer containing a thin film transistor (TFT), an OLED display element, a barrier film, a color filter, glass, a pressure-sensitive adhesive layer, the optical filter according to the embodiment of the present invention, and a surface film, in order from the opposite side to external light.

The OLED display element has a configuration in which an anode electrode, a light emitting layer, and a cathode electrode are laminated in this order. In addition to the light emitting layer, a hole injection layer, a hole transport layer, an electron transport layer, an electron injection layer, and the like are included between the anode electrode and the cathode electrode. In addition, for example, the description in JP2014-132522A can also be referenced.

In addition, as the color filter, in addition to a typical color filter, a color filter in which quantum dots are laminated can also be used.

A resin film can be used instead of the above glass.

### <Pressure-Sensitive Adhesive Layer>

In the OLED display device according to the embodiment of the present invention, a surface of the optical filter according to the embodiment of the present invention on the external light side may be bonded to an optical functional film having an antireflection layer or the like, or a polarizing plate including a polarizer and a polarizing plate protective film while interposing a pressure-sensitive adhesive layer. In addition, it is preferable that a surface of the optical filter according to the embodiment of the present invention, which is positioned opposite to the side of the external light, is bonded to the glass (the base material) with a pressure-sensitive adhesive layer being interposed.

For the pressure-sensitive adhesive layer, the descriptions related to the pressure-sensitive adhesive layer and the forming method in the OLED display device, which are described in [0239] to [0290] of WO2021/132674A, can be applied as they are.

It is noted that the pressure-sensitive adhesive composition described in WO2021/132674A preferably contains the above-described ultraviolet absorbing agent from the viewpoint of the light resistance of the optical filter.

### <Base Material>

In the OLED display device according to the embodiment of the present invention, a surface of the optical filter according to the embodiment of the present invention, which is positioned on the external light side, may be bonded to an optical functional film with a pressure-sensitive adhesive layer being interposed. In addition, it is preferable that a surface of the optical filter according to the embodiment of the present invention, which is positioned opposite to the side of the external light, is bonded to the glass (the base material) with a pressure-sensitive adhesive layer being interposed.

The method of forming the adhesive layer is not particularly limited, and for example, a method of applying the pressure-sensitive adhesive composition to the light absorption filter or optical filter according to the embodiment of the present invention by a usual means such as a bar coater, drying, and curing the pressure-sensitive adhesive composition; a method of applying the pressure-sensitive adhesive composition first to the surface of a peelable base material, and drying the composition, and then transferring the pressure-sensitive adhesive layer using the peelable base material to the light absorption filter according to the embodiment of the present invention and then aging and curing the composition is used.

The peelable base material is not particularly limited, and a predetermined peelable base material can be used. Examples thereof include the support film in the manufacturing method for the light absorption filter according to the embodiment of the present invention described above.

In addition, the conditions of application, drying, aging, and curing can be appropriately adjusted based on a conventional method.

### [Inorganic Electroluminescent Display Device]

An inorganic electroluminescent display device (referred to as an inorganic EL (electroluminescence) display device and also simply referred to as an inorganic EL display device in the present invention) according to an embodiment of the present invention includes the optical filter according to the embodiment of the present invention.

As another configuration of the inorganic EL display device according to the embodiment of the present invention, a configuration of a generally used inorganic EL display device can be used without particular limitation as long as the optical filter according to the embodiment of the present invention is included. For example, the descriptions regarding the inorganic EL element and the inorganic electroluminescent display device, which are described in JP2005-338640A can be preferably applied.

### [Liquid Crystal Display Device]

The liquid crystal display device according to the embodiment of the present invention includes the optical filter according to the embodiment of the present invention.

The optical filter according to the embodiment of the present invention may be used as at least one of a polarizing plate-protective film or a pressure-sensitive adhesive layer as described later, or it may be included in a backlight unit that is used in the liquid crystal display device.

It is preferable that the liquid crystal display device includes the optical filter according to the embodiment of the present invention, a polarizing plate including a polarizer and a polarizing plate-protective film, a pressure-sensitive adhesive layer, and a liquid crystal cell, where it is preferable that the polarizing plate is bonded to the liquid crystal cell with a pressure-sensitive adhesive layer being interposed. In the liquid crystal display device, the optical filter according to the embodiment of the present invention may also serve as the polarizing plate-protective film or the pressure-sensitive adhesive layer. That is, the liquid crystal display device is divided into a case where the liquid crystal display device includes a polarizing plate including a polarizer and the optical filter (polarizing plate-protective film) according to the embodiment of the present invention, a pressure-sensitive adhesive layer, and a liquid crystal cell, and a case where the liquid crystal display device includes a polarizing plate including a polarizer and a polarizing plate-protective film, the optical filter (pressure-sensitive adhesive layer) according to the embodiment of the present invention, and a liquid crystal cell.

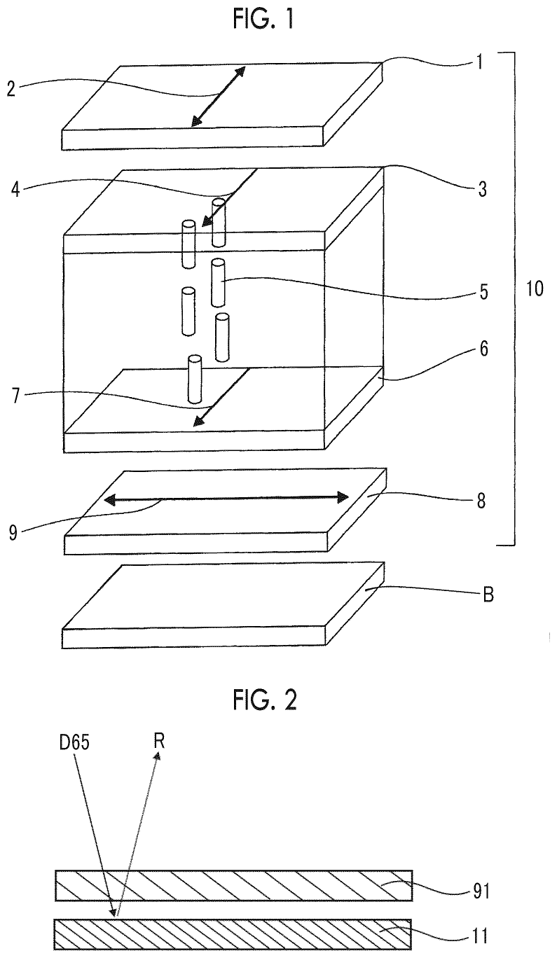

FIG. 1 is a schematic view illustrating an example of the liquid crystal display device according to the embodiment of the present invention. In FIG. 1, a liquid crystal display device 10 consists of a liquid crystal cell having a liquid crystal layer 5 and having a liquid crystal cell upper electrode substrate 3 and a liquid crystal cell lower electrode substrate 6, which are respectively disposed above and below the liquid crystal layer 5, and an upper polarizing plate 1 and a lower polarizing plate 8, which are respectively disposed on both sides of the liquid crystal cell. A color filter layer may be laminated on the upper electrode substrate 3 or the lower electrode substrate 6. On the rear surface of the liquid crystal display device 10, a backlight is disposed. As a light source of the backlight, those described in the above backlight unit can be used.

Each of the upper polarizing plate 1 and the lower polarizing plate 8 has a configuration in which each of them is laminated such that a polarizer is sandwiched between two polarizing plate protective films, and in the liquid crystal display device 10, at least one polarizing plate is preferably a polarizing plate including the optical filter according to the embodiment of the present invention.

In addition, in the liquid crystal display device 10, the liquid crystal cell may be bonded to the polarizing plates (the upper polarizing plate 1 and/or the lower polarizing plate 8) with a pressure-sensitive adhesive layer (not illustrated in the drawing) being interposed. In this case, the optical filter according to the embodiment of the present invention may also serve as the above-described pressure-sensitive adhesive layer.

The liquid crystal display device 10 includes an image direct vision-type liquid crystal display, an image projection-type liquid crystal display device, and a light modulation-type liquid crystal display device. The present invention is effective for an active matrix liquid crystal display device that uses a three-terminal or two-terminal semiconductor element such as a thin film transistor (TFT) or a metal insulator metal (MIM). Of course, it is also effective for a passive matrix liquid crystal display device represented by a super twisted nematic (STN) mode which is called the time division driving.

In a case where the optical filter according to the embodiment of the present invention is included in the backlight unit, the polarizing plate of the liquid crystal display device may be a general polarizing plate (a polarizing plate that does not include the optical filter according to the embodiment of the present invention) or may be a polarizing plate that includes the optical filter according to the embodiment of the present invention. In addition, the pressure-sensitive adhesive layer may be a typical pressure-sensitive adhesive layer (not the optical filter according to the embodiment of the present invention) or may be a pressure-sensitive adhesive layer formed of the optical filter according to the embodiment of the present invention.

The in-plane switching (IPS) mode liquid crystal display device described in paragraphs 0128 to 0136 of JP2010-102296A is preferable as the liquid crystal display device according to the embodiment of the present invention except that the optical filter according to the embodiment of the present invention is used.

### <Polarizing Plate>

The polarizing plate that is used in the present invention includes a polarizer and at least one polarizing plate-protective film.

The polarizing plate that is used in the present invention is preferably a polarizing plate having a polarizer and polarizing plate-protective films on both surfaces of the polarizer, and it is preferable that at least one surface of the polarizer includes the optical filter according to the embodiment of the present invention as the polarizing plate-protective film. In this case, the surface of the polarizer opposite to the surface having the optical filter according to the embodiment of the present invention (the polarizing plate-protective film according to the embodiment of the present invention) may have a general polarizing plate-protective film.

The film thickness of the polarizing plate protective film is preferably 5 to 120 μm and more preferably 10 to 100 μm. A thinner film is preferable since in a case of being incorporated in the liquid crystal display device, the display unevenness after elapse of time in high temperature and high humidity is less likely to occur. On the other hand, in a case where the film is too thin, it is difficult to transport the film stably at the time of producing the film and producing the polarizing plate. In a case where the optical filter according to the embodiment of the present invention also serves as the polarizing plate-protective film, it is preferable that the thickness of the optical filter satisfies the above-described range.

For the polarizing plate that is used in the present invention, the descriptions related to the performance, the shape, the configuration, the polarizer, the method of laminating the polarizer and the polarizing plate-protective film, the functionalization of the polarizing plate, and the like regarding the polarizing plate described in [0299] to [0309] of WO2021/132674A can be applied as they are.

### <Pressure-Sensitive Adhesive Layer>

In the liquid crystal display device according to the embodiment of the present invention, the polarizing plate is preferably bonded to the liquid crystal cell with a pressure-sensitive adhesive layer being interposed. The optical filter according to the embodiment of the present invention may also serve as the pressure-sensitive adhesive layer. In a case where the optical filter according to the embodiment of the present invention does not serve as the pressure-sensitive adhesive layer, a typical pressure-sensitive adhesive layer can be used as the pressure-sensitive adhesive layer.

The pressure-sensitive adhesive layer is not particularly limited as long as the polarizing plate can be bonded to the liquid crystal cell, and for example, an acrylic type, a urethane type, polyisobutylene, or the like is preferable.

In a case where the optical filter according to the embodiment of the present invention also serves as a pressure-sensitive adhesive layer, the pressure-sensitive adhesive layer includes the dye and the base polymer, and further contains a crosslinking agent, a coupling agent, or the like to impart adhesiveness.

In a case where the optical filter according to the embodiment of the present invention also serves as a pressure-sensitive adhesive layer, the pressure-sensitive adhesive layer preferably contains 90% to 100% by mass of the base polymer, and preferably contains 95% to 100% by mass of the base polymer. The content of the coloring agent is as described above.

The thickness of the pressure-sensitive adhesive layer is not particularly limited; however, it is preferably 1 to 50 μm and more preferably 3 to 30 μm.

### <Liquid Crystal Cell>

The liquid crystal cell is not particularly limited, and a typical liquid crystal cell can be used.

### <Ultraviolet Absorbing Layer>

The organic electroluminescent display device, inorganic electroluminescent display device, or liquid crystal display device including the optical filter according to the embodiment of the present invention preferably has a layer (hereinafter, also referred to as an “ultraviolet absorbing layer”) that inhibits the light absorption (the ultraviolet absorption) of the compound that generates a radical upon ultraviolet irradiation, on the viewer side with respect to the optical filter according to the embodiment of the present invention. In a case where the ultraviolet absorbing layer is provided, it is possible to prevent the fading of the optical filter according to the embodiment of the present invention due to external light.

Hereinafter, the ultraviolet absorbing layer that is used in the present invention will be described.

### (Ultraviolet Absorbing Agent)

The ultraviolet absorbing layer usually contains a resin and an ultraviolet absorbing agent.

Specific examples of the ultraviolet absorbing agent preferably used in the present invention include a hindered phenol-based compound, a benzophenone-based compound such as a hydroxybenzophenone-based compound, a benzotriazole-based compound, a salicylic acid ester-based compound, a cyanoacrylate-based compound, and a nickel complex salt-based compound.

Examples of the hindered phenol-based compound include 2,6-di-tert-butyl-p-cresol, pentaerythrityl-tetrakis[3-(3,5-di-tert-butyl-4-hydroxyphenyl)propionate], N,N′-hexamethylene bis(3,5-di-tert-butyl-4-hydroxy-hydrocinnamide), 1,3,5-trimethyl-2,4,6-tris(3, 5-di-tert-butyl-4-hydroxybenzyl)benzene, and tris-(3, 5-di-tert-butyl-4-hydroxybenzyl)-isocyanurate.

Examples of the benzotriazole-based compound include 2-(2′-hydroxy-5′-methylphenyl) benzotriazole, 2,2-methylene bis(4-(1,1,3,3-tetramethylbutyl)-6-(2H-benzotriazole-2-yl) phenol), 2,4-bis(n-octylthio)-6-(4-hydroxy-3,5-di-tert-butylanilino)-1,3,5-triazine, triethyleneglycol-bis[3-(3-tert-butyl-5-methyl-4-hydroxyphenyl)propionate], N,N′-hexamethylene bis(3,5-di-tert-butyl-4-hydroxy-hydrocinnamide), 1,3,5-trimethyl-2,4,6-tri s(3,5-di-tert-butyl-4-hydroxybenzyl)benzene, 2-(2′-hydroxy-3′, 5′-Di-tert-butylphenyl)-5-chlorbenzotriazole, 2-(2′-hydroxy-3′, 5′-di-tert-amylphenyl)-5-chlorbenzotriazole, 2,6-di-tert-butyl-p-cresol, and pentaerythrityl-tetrakis[3-(3,5-di-tert-butyl-4-hydroxyphenyl)propionate].

The adding amount of this ultraviolet absorbing agent is preferably 0.1 parts by mass to 30.0 parts by mass with respect to 100 parts by mass of the resin.

In addition, from the viewpoint of further improving the light resistance of the optical filter according to the embodiment of the present invention, it is also preferable to use the compound (1) represented by Formula (1) described in paragraphs [0229] to [0283] of WO2023/068235A as the ultraviolet absorber. For the absorption characteristics, the synthesis method, and the content of the compound (1) represented by Formula (1) described in WO2023/068235A, the description in paragraphs 0279 to 0283 of WO2023/068235A can also be suitably used in the present invention.

### (Resin)

As the resin that is used for the ultraviolet absorbing layer, a known resin can be used, which is not particularly limited as long as it does not contradict the gist of the present invention. Examples of the resin include a cellulose acylate resin, an acrylic resin, a cycloolefin-based resin, a polyester-based resin, and an epoxy resin.

### (Installation Position of Ultraviolet Absorbing Layer)

The disposition of the ultraviolet absorbing layer is not particularly limited as long as it is on the viewer side with respect to the optical filter according to the embodiment of the present invention, and the ultraviolet absorbing layer can be installed at any position. For example, it is also possible to add an ultraviolet absorbing agent to a member such as a protective film of the polarizing plate, an antireflection film, or the like to impart it a function of an ultraviolet absorbing layer. In addition, an ultraviolet absorbing agent can also be added to the above-described pressure-sensitive adhesive layer.

### EXAMPLES

Hereinafter, the present invention will be described in more detail based on Examples. The materials, using amount, ratio, details of treatment, procedures of treatment, and the like described in Examples below can be appropriately changed without departing from the spirit of the present invention. Therefore, it is to be understood that the scope of the present invention is not limited to Examples described below.

It is noted that “parts” and “%” that indicate the composition in Examples below are based on mass unless otherwise specified.

All steps from a preparation step of a light absorption filter forming liquid to a production step of a light absorption filter using the light absorption filter forming liquid and to the use thereof in the ultraviolet irradiation test were carried out under a yellow lamp to avoid ultraviolet irradiation.

### [Production of Light Absorption Filter]

Materials used to produce the light absorption filter are shown below.

### <Matrix Polymer (Resin)>

### (Resin 1)

A cyclohexyl methacrylate-methacrylic acid random copolymer, methacrylic acid content: 29% by mole, weight-average molecular weight: 26300.

It is noted that the methacrylic acid moiety of the resin 1 corresponds to the compound A having an acid group defined in the present invention.

### <Compound B>

4-methylquinoline (manufactured by Tokyo Chemical Industry Co., Ltd., Lepidine, pKaH: 5.1)

### <Dye>

In the following structural formulae, Me represents a methyl group, Ac represents an acetyl group, Ph represents a phenyl group, and C₈H₁₇ represents an n-octyl group.

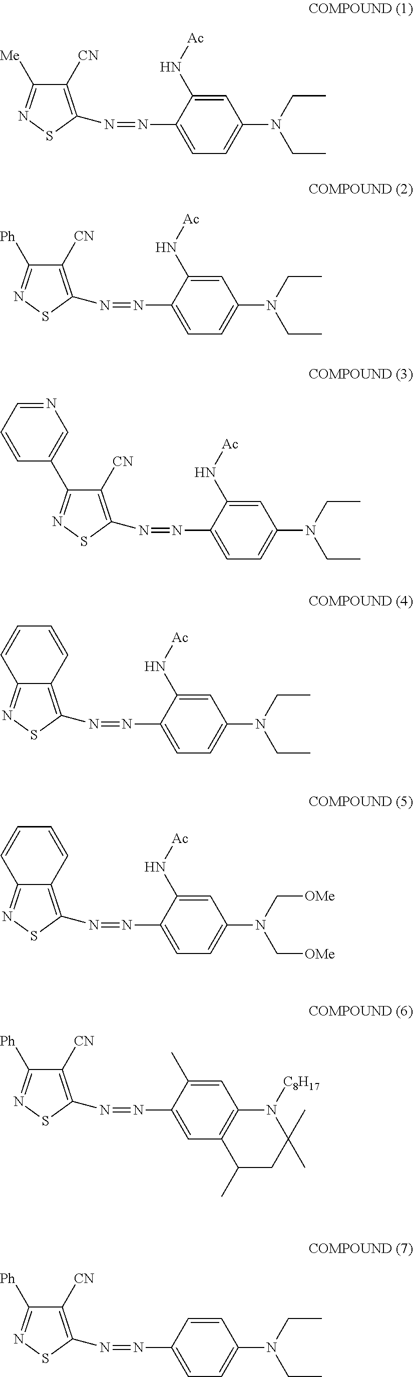

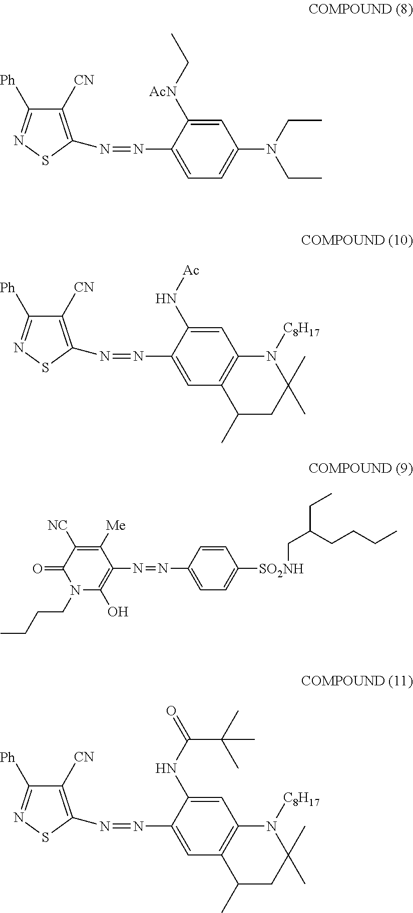

The compounds (1) to (5), (10), and (11) were synthesized by the azo coupling of the above-described amino-substituted isothiazole derivative and the amide-substituted aniline derivative. The ¹H-NMR results of each compound are as follows.

### <NMR Data of Compound (1)>

¹H-NMR (CDCl₃-d) δ=1.30-1.34 (t, 6H), 2.31 (s, 0.9H), 2.37 (s, 2.1H), 2.60-2.63 (s, 3H), 3.53-3.59 (q, 4H), 6.49 (d, 0.3H), 6.56 (d, 0.7H), 7.54 (d, 0.7H), 7.94 (d, 0.3H), 8.07 (s, 0.3H), 8.30 (s, 0.7H), 9.04 (s, 0.3H), 12.6 (s, 0.7H)

### <NMR Data of Compound (2)>

¹H-NMR (CDCl₃-d) δ=1.31-1.35 (t, 6H), 2.31 (s, 0.7H), 2.40 (s, 2.3H), 3.55-3.60 (q, 4H), 6.52 (m, 0.3H), 6.57-6.60 (d, 0.7H), 7.49-7.57 (m, 3H), 7.98 (d, 0.3H), 8.07 (s, 2H), 8.33 (s, 0.7H), 9.07 (s, 0.3H), 12.5 (s, 0.7H)

### <NMR Data of Compound (3)>

¹H-NMR (CDCl₃-d) δ=1.31-1.35 (t, 6H), 2.31 (s, 0.7H), 2.40 (s, 2.3H), 3.56-3.61 (q, 4H), 6.51-6.53 (d, 0.2H), 6.59-6.61 (d, 0.8H), 7.44-7.47 (m, 1H), 7.52-7.57 (d, 0.8H), 7.98-8.00 (d, 0.2H), 8.08 (m, 0.3H), 8.34-8.37 (d, 0.7H), 8.37-8.40 (d, 1H), 8.72-8.74 (d, 2H), 9.05 (s, 0.2H), 9.30 (s, 0.3H), 9.32 (s, 0.7H), 12.6 (s, 0.7H)

### <NMR Data of Compound (4)>

¹H-NMR (CDCl₃-d) δ=1.25-1.32 (t, 6H), 2.35 (s, 3H), 3.50-3.56 (q, 4H), 6.50-6.53 (m, 1H), 7.22-7.25 (m, 2H), 7.41-7.45 (m, 1H), 7.68-7.71 (d, 1H), 7.83 (brs, 1H), 8.06-8.08 (d, 1H), 8.13 (s, 1H).

### <NMR Data of Compound (5)>

¹H-NMR (CDCl₃-d) δ=2.35 (s, 3H), 3.38 (s, 6H), 3.64-3.67 (t, 4H), 3.74-3.77 (t, 4H), 6.59-6.62 (m, 1H), 7.24-7.28 (m, 1H), 7.41-7.56 (m, 1H), 7.70-7.72 (d, 1H), 7.83-7.86 (d, 1H), 8.07-8.09 (d, 1H), 8.15 (s, 1H).

### <NMR Data of Compound (10)>

¹H-NMR (CDCl₃-d) δ=0.88-0.91 (m, 3H), 1.25-1.86 (m, 23H), 2.30 (s, 0.7H), 2.39 (s, 2.3H), 2.84-2.87 (m, 1H), 3.34-3.42 (m, 1H), 3.46-3.54 (m, 1H), 7.36 (s, 0.8H), 7.48-7.54 (m, 3H), 7.83 (s, 0.2H), 7.99 (s, 0.3H), 8.07-8.09 (m, 2H), 8.25 (s, 0.7H), 9.05 (s, 0.2H), 12.7 (s, 0.8H)

### <NMR Data of Compound (11)>

¹H-NMR (CDCl₃-d) δ=0.86-0.91 (m, 3H), 1.25-1.86 (m, 32H), 2.82-2.86 (m, 1H), 3.37-3.44 (m, 1H), 3.48-3.56 (m, 1H), 7.36 (s, 0.3H), 7.49-7.54 (m, 3H), 7.87 (s, 0.7H), 7.99 (s, 0.7H), 8.04-8.09 (m, 2H), 8.32 (s, 0.3H), 9.45 (s, 0.7H), 12.0 (s, 0.3H)

### (Leveling Agent 1)

A polymer surfactant composed of the following constitutional components was used as a leveling agent 1. In the following structural formulae, the proportion of each constitutional component is in terms of a molar ratio, and t-Bu means a tert-butyl group.

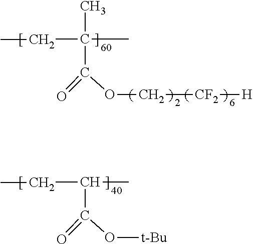

### (Base Material 1)

A polyethylene terephthalate film (manufactured by TORAY INDUSTRIES, Inc., product name: Lumirror XD-510P, film thickness: 50 m)

### EXAMPLES

### <1. Production of Light Absorption Filter No. 101>

### (1) Preparation of Resin Solution (Light Absorption Filter Forming Liquid)

Each component was mixed with the composition shown below to prepare a light absorption filter forming liquid (composition) 1.

Composition of light absorbing filter forming liquid 1

| | |
|---|---|
| Resin 1 | 81.1 parts by mass |
| Leveling agent 1 | 0.08 parts by mass |
| Compound (9) | 2.73 parts by mass |
| Compound (1) | 2.12 parts by mass |
| Compound B (4-methylquinoline | 17.2 parts by mass |
| (manufactured by Tokyo Chemical | |
| Industry Co., Ltd.)) | |
| Methyl ethyl ketone (solvent) | 566.7 parts by mass |

Subsequently, the obtained light absorption filter forming liquid (composition) 1 was filtered using a filter paper (#63, manufactured by Toyo Roshi Kaisha, Ltd.) having an absolute filtration precision of 10 μm, and further subjected to filtration using a metal sintered filter (product name: Pall filter PMF, media code: FH025, manufactured by Pall) with an absolute filtration precision of 2.5 μm.

### (2) Production of Light Absorption Filter

The light absorption filter forming liquid (composition) 1 after the filtration treatment was applied onto the base material 1 by using a bar coater so that the film thickness after drying was 2.2 μm, and dried at 120° C to produce a light absorption filter No. 101.

<2. Production of Light Absorption Filters Nos. 102 to 107 and c11 to c13>

Light absorption filters Nos. 102 to 107 and c11 to c13 were produced in the same manner as in the production of the light absorption filter No. 101, except that the type and the blending amount of the dye were changed to the contents shown in Table 1 in the production of the light absorption filter No. 101. It is noted that the adjustment is carried out such that the blending amount of the leveling agent 1 and the compound B in the light absorption filter No. 101 is fixed, the blending amount of the resin is changed according to the change in the blending amount of the dye, and the mass of the entire filter does not change.

In addition, a light absorption filter No. r201 was produced in the same manner, except that in the production of the light absorption filter No. 101, the compound B and the dye were not blended and the blending amount of the resin was changed so that the mass of the entire filter was not changed.

Here, the light absorption filters Nos. 101 to 107 are the light absorption filters according to the embodiment of the present invention, the light absorption filters Nos. c11 to c13 are light absorption filters for comparison, and the light absorption filter No. r201 is a reference light absorption filter.

### [Production of Light Absorption Filter Having Gas Barrier Layer]

Regarding each of the light absorption filters Nos. 101 to 107, c11 to c13, and r201, a light absorption filter (a light absorption filter having a gas barrier layer) formed by further laminating a gas barrier layer on the light absorption filter was produced as described below, and the evaluation described later was carried out.

### (1) Production of Base Material 3

The light absorption filter side (side opposite to the base material 1) of the light absorption filter having the base material produced as described above was subjected to a corona treatment using a corona treatment device (trade name: Corona-Plus, manufactured by VETAPHONE GmbH) under conditions of a discharge amount of 1,000 W min/m² and a treatment speed of 3.2 m/min, and used as a base material 3.

### (2) Preparation of Resin Solution

Each component was mixed with the composition shown below, and the resultant mixture was stirred in a constant-temperature tank at 90° C for 1 hour to dissolve Kuraray Exceval AQ-4105 (product name, manufactured by KURARAY Co., Ltd., modified polyvinyl alcohol, saponification degree: 98 to 99 mol %), whereby a gas barrier layer forming liquid was prepared.

Composition of gas barrier layer forming liquid

| | |
|---|---|
| Kuraray Exceval AQ-4105 (product | 4.0 parts by mass |
| name, manufactured by KURARAY | |
| Co., Ltd.) | |
| Pure water | 88.5 parts by mass |
| Isopropyl alcohol | 7.5 parts by mass |

Subsequently, the obtained gas barrier layer forming liquid was filtered using a filter having an absolute filtration precision of 5 μm (product name: Hydrophobic Fluorepore Membrane, manufactured by Millex).

### (3) Lamination of Gas Barrier Layer

The gas barrier layer forming liquid after the filtration treatment was applied to the corona-treated surface side of the base material 3 using a bar coater so that the film thickness after drying was 1.6 μm, and dried at 120° C for 60 seconds, whereby a light absorption filter having a gas barrier layer was produced.

The light absorption filter having a gas barrier layer has a configuration in which the base material 1, the light absorption filter, and the gas barrier layer are laminated in this order.

### <Evaluation of Physical Properties of Gas Barrier Layer>

The physical properties of the gas barrier layer, which had been measured by the method described in [0182] to [0184] of WO2022/149510A, were a degree of crystallinity of 53%, an oxygen permeability of 0.4 cc/m²·day·atm, and a thickness of 1.6 μm, respectively.

### <Absorbance of Light Absorption Filter (Before Ultraviolet Irradiation)>

### (1) Measurement of Absorbance

Using a UV3600 spectrophotometer (product name) manufactured by Shimadzu Corporation, the absorbance of the light absorption filter having a gas barrier layer and the standard filter in a wavelength range of 380 to 800 nm was measured for every 1 nm.

A standard filter for the light absorption filters Nos. 101 to 107, and c11 to c13, which contain the resin 1, is the light absorption filter No. r201 which has been changed not to contain the dye and the compound B.

### (2) Calculation of Absorbance

Using the absorbance value Abx(X) of the light absorption filter having a gas barrier layer at each wavelength X nm measured as described above and the absorbance value Abo(X) of the standard filter containing the same resin at each wavelength X nm, the absorbance Ab(X) of the light absorption filter before ultraviolet irradiation was calculated according to the following expression.

Ab(λ)=Abₓ(λ)−Ab₀(λ)

Hereinafter, among the absorbances Ab(λ) of the light absorption filter in a wavelength range of 380 to 800 nm, the wavelength at which the highest absorbance Ab(λ) among the wavelengths at which the highest maximal absorption is exhibited was defined as the maximal absorption wavelength (hereinafter, also simply referred to as “λₘₐₓ”), and the absorbance at λₘₐₓ was defined as the absorption maximal value (hereinafter, also simply referred to as “Ab(λₘₐₓ)”).

### <<Evaluation>>

For each light absorption filter, the tint of the reflected light, the decolorization properties (decolorization rate and the presence or absence of secondary absorption due to decomposition of the coloring agent), and the heat resistance were evaluated.

The results are summarized and shown in Tables 2 and 3 (Tables 3A and 3B are collectively referred to as “Table 3”).

### [1. Evaluation of Tint of Reflected Light]

### <Simulation of Tint of Reflected Light>

For the light absorption filter before the gas barrier layer was laminated as described above, a simulation related to external light reflection on the aluminum foil substrate was performed in a configuration in which the light absorption filter was transferred to the aluminum foil substrate through a pressure-sensitive adhesive and the base material 1 was peeled off, and the tint (a* and b*) of the reflected light was calculated.

Specifically, in the configuration shown in FIG. 2, the intensity of light (reflected light R) in a case where white light having a spectrum of a standard illuminant D65 defined by the International Commission on Illumination (CIE) was transmitted through a light absorption filter 91, reflected by an aluminum foil substrate 11 having a reflectivity of 85%, and transmitted through the light absorption filter 91 again was calculated for each 1 nm in a wavelength range of 380 to 780 nm, and the tint (a*, b*) was calculated by multiplying the intensity by a standard photopic luminosity and adding the products (correcting the photopic sensitivity).

From the viewpoint of bringing the tint of the reflected light close to neutral (adjusting the tint), both a* and b* are preferably close to 0 as an absolute value, and for example, a level of 13 or less is preferable and a level of 4 or less is more preferable.

### (Ultraviolet Irradiation Test for Evaluation of Decolorization Properties and Heat Resistance)

The light absorption filter having a gas barrier layer and the standard filter were irradiated with ultraviolet rays (UV) having a predetermined irradiation amount at an illuminance of 100 mW/cm² from the gas barrier layer side (side opposite to the base material 1) under conditions of 35° C using an ultra-high pressure mercury lamp (manufactured by HOYA Corporation, trade name: UL750) under atmospheric pressure (101.33 kPa).

### [2-1. Evaluation of Decolorization Rate]

The decolorization rate was calculated according to the following expression using the absorption maximal values (Ab(λₘₐₓ)) before and after the ultraviolet irradiation test.

Decolorization rate (%)=100−(Ab(λₘₐₓ) after ultraviolet irradiation/Ab(λₘₐₓ) before ultraviolet irradiation)×100%

It is noted that the ultraviolet irradiation was performed by setting the ultraviolet irradiation amount to 1330 mJ/cm² in the above-described ultraviolet irradiation test.

[2-2. Evaluation of Presence or Absence of Secondary Absorption Associated with Decomposition of Coloring Agent]

The presence or absence of the absorption (the secondary absorption) derived from a new coloration structure associated with the decomposition of the coloring agent was evaluated based on the ratio of the absorbance at a wavelength of 650 nm to the absorption maximum value (Ab(λₘₐₓ)) before ultraviolet irradiation (hereinafter, also simply referred to as “Ab(650)”). It is meant that the smaller the value obtained by subtracting the ratio of the following (IV) from the ratio of the following (III), the less frequently the absorption derived from the new coloration structure associated with the decomposition of the coloring agent occurs.

{Ab(650) before ultraviolet irradiation/Ab(λₘₐₓ) before ultraviolet irradiation}×100% (III)

{Ab(650) after ultraviolet irradiation/Ab(λₘₐₓ) before ultraviolet irradiation}×100% (IV)

It is noted that the ultraviolet irradiation was performed by setting the ultraviolet irradiation amount to 1330 mJ/cm² in the above-described ultraviolet irradiation test.

### [3. Evaluation of Heat Resistance]

The heat resistance under high humidity conditions was evaluated by a heat resistance test in which the light absorption filter (filter after UV decolorization) after ultraviolet irradiation was allowed to stand for 168 hours in an environment of 60° C and a relative humidity of 90%, and the change in the transmittance at 400 nm, 500 nm, and 600 nm before and after the heat resistance test.

In addition, the heat resistance in a dry environment was evaluated by a heat resistance test in which the light absorption filter (filter after UV decolorization) after ultraviolet irradiation was allowed to stand for 168 hours in a dry environment (a state in which the humidity was not adjusted, which means a relative humidity of 20%) at 80° C, and the change in the transmittance at 400 nm, 500 nm, and 600 nm before and after the heat resistance test.

It is noted that the ultraviolet irradiation was performed by setting the ultraviolet irradiation amount to 3000 mJ/cm² in the above-described ultraviolet irradiation test.

It is noted that each transmittance at 400 nm, 500 nm, and 600 nm was calculated from the following calculation expression using the absorbance measured by the method described in the measurement of the absorbance described above.

Absorbance=−log₁₀(T/100)

TABLE 1

| Light | Dye | | | Compound B | |
|---|---|---|---|---|---|
| absorption | | Blending | | | Blending |
| filter No. | Type | amount | Resin | Type | amount |
| 101 | Compound (9) | 2.73 parts | 1 | 4- | 17.2 parts |
| | Compound (1) | 2.12 parts | | methylquinoline | |
| 102 | Compound (9) | 2.73 parts | 1 | 4- | 17.2 parts |
| | Compound (2) | 2.48 parts | | methylquinoline | |
| 103 | Compound (9) | 2.73 parts | 1 | 4- | 17.2 parts |
| | Compound (3) | 2.49 parts | | methylquinoline | |
| 104 | Compound (9) | 2.73 parts | 1 | 4- | 17.2 parts |
| | Compound (4) | 2.18 parts | | methylquinoline | |
| 105 | Compound (9) | 2.73 parts | 1 | 4- | 17.2 parts |
| | Compound (5) | 2.37 parts | | methylquinoline | |
| 106 | Compound (9) | 2.73 parts | 1 | 4- | 17.2 parts |
| | Compound (10) | 3.31 parts | | methylquinoline | |
| 107 | Compound (9) | 2.73 parts | 1 | 4- | 17.2 parts |
| | Compound (11) | 3.56 parts | | methylquinoline | |
| c11 | Compound (9) | 2.73 parts | 1 | 4- | 17.2 parts |
| | Compound (6) | 3.05 parts | | methylquinoline | |
| c12 | Compound (9) | 2.73 parts | 1 | 4- | 17.2 parts |
| | Compound (7) | 2.15 parts | | methylquinoline | |
| c13 | Compound (9) | 2.73 parts | 1 | 4- | 17.2 parts |
| | Compound (8) | 2.49 parts | | methylquinoline | |

TABLE 2

| | | | | | | | | | Ratio of Ab(650) to | |
|---|---|---|---|---|---|---|---|---|---|---|
| | | | | | | | | | Ab(max) before | |
| | | | | | Tint of | | | | ultraviolet irradiation | |
| Light | | | | | reflected | | Decolorization | | Before | After |
| absorption | λmax | | Ab(λmax) | | light | | rate | | ultraviolet | ultraviolet |
| filter No. | (i) | (ii) | (i) | (ii) | a* | b* | (i) | (ii) | irradiation | irradiation |
| 101 | 431 nm | 558 nm | 0.62 | 0.80 | 31.1 | 13.0 | 72% | 84% | 0% | 0% |
| 102 | 431 nm | 564 nm | 0.62 | 0.89 | 33.4 | 3.9 | 70% | 84% | 0% | 0% |
| 103 | 431 nm | 567 nm | 0.62 | 0.89 | 30.3 | 28.3 | 70% | 88% | 0% | 0% |
| 104 | 431 nm | 575 nm | 0.62 | 0.70 | −11.1 | 1.1 | 61% | 81% | 9% | 2% |
| 105 | 431 nm | 570 nm | 0.62 | 0.67 | −3.2 | 3.2 | 59% | 81% | 6% | 1% |
| 106 | 431 nm | 581 nm | 0.62 | 0.95 | 12.8 | −2.2 | 63% | 79% | 1% | 0% |
| 107 | 431 nm | 584 nm | 0.62 | 1.01 | 4.1 | −1.9 | 65% | 79% | 3% | 1% |
| c11 | 431 nm | 579 nm | 0.62 | 0.83 | 2.9 | −2.5 | 68% | 77% | 1% | 0% |
| c12 | 431 nm | 556 nm | 0.62 | 0.75 | 27.6 | 8.0 | 73% | 91% | 2% | 0% |
| c13 | 431 nm | 556 nm | 0.62 | 0.70 | 28.8 | 9.2 | 76% | 95% | 1% | 0% |

TABLE 3A
Heat resistance test of filter after UV
decolorization under conditions of 80 °C,
dry environment, and 168 hours

| | Trans- | | | Trans- | | | Change | | |
|---|---|---|---|---|---|---|---|---|---|
| | mittance T_{B} | | | mittance | | | (T_{B} − T_{A}) of | | |
| | before heat | | | T_{A} after heat | | | transmittance | | |
| | resistance | | | resistance | | | by heat | | |
| Light | test | | | test | | | resistance test | | |
| absorption | 400 | 500 | 600 | 400 | 500 | 600 | 400 | 500 | 600 |
| filter No. | nm | nm | nm | nm | nm | nm | nm | nm | nm |
| 101 | 95 | 100 | 100 | 92 | 97 | 100 | 3 | 3 | 0 |
| 102 | 94 | 100 | 100 | 91 | 97 | 99 | 3 | 3 | 1 |
| 103 | 94 | 100 | 100 | 90 | 98 | 99 | 4 | 2 | 1 |
| 104 | 85 | 99 | 100 | 85 | 98 | 100 | 0 | 1 | 0 |
| 105 | 85 | 100 | 100 | 85 | 98 | 100 | 0 | 2 | 0 |
| 106 | 94 | 100 | 100 | 91 | 97 | 100 | 3 | 3 | 0 |
| 107 | 93 | 100 | 100 | 89 | 97 | 99 | 4 | 3 | 1 |
| c11 | 93 | 99 | 99 | 83 | 93 | 98 | 10 | 6 | 1 |
| c12 | 93 | 97 | 99 | 86 | 94 | 98 | 7 | 3 | 1 |
| c13 | 95 | 99 | 100 | 90 | 93 | 97 | 5 | 6 | 3 |

TABLE 3B
Heat resistance test of filter after UV
decolorization under conditions of 60 °C, 90%
relative humidity environment, and 168 hours

| | | | | | | | Change | | |
|---|---|---|---|---|---|---|---|---|---|
| | | | | | | | (T_{B} − T_{A}) of | | |
| | Transmittance | | | Transmittance | | | transmittance | | |
| | T_{B} before heat | | | T_{A} after heat | | | by heat | | |
| Light | resistance test | | | resistance test | | | resistance test | | |
| absorption | 400 | 500 | 600 | 400 | 500 | 600 | 400 | 500 | 600 |
| filter No. | nm | nm | nm | nm | nm | nm | nm | nm | nm |
| 101 | 94 | 100 | 100 | 90 | 98 | 100 | 4 | 2 | 0 |
| 102 | 94 | 100 | 100 | 92 | 98 | 100 | 2 | 2 | 0 |
| 103 | 94 | 100 | 100 | 90 | 97 | 99 | 4 | 3 | 1 |
| 104 | 85 | 99 | 100 | 85 | 98 | 100 | 0 | 1 | 0 |
| 105 | 85 | 99 | 100 | 85 | 97 | 100 | 0 | 2 | 0 |
| 106 | 94 | 100 | 100 | 91 | 98 | 100 | 3 | 2 | 0 |
| 107 | 93 | 100 | 100 | 89 | 98 | 99 | 4 | 2 | 1 |
| c11 | 93 | 99 | 99 | 83 | 93 | 97 | 10 | 6 | 2 |
| c12 | 93 | 97 | 99 | 85 | 94 | 97 | 8 | 3 | 2 |
| c13 | 95 | 99 | 100 | 87 | 97 | 99 | 8 | 2 | 1 |

(Note in Table 1)
The dye, the resin, and the compound B are as described above for the dye, the resin, and the compound B, respectively. The blending amounts of the dye and the compound B mean the blending amounts (parts by mass) in the above-described resin solution (light absorption filter forming liquid).
(Note in Table 2)

λₘₐₓ means a maximal absorption wavelength of the light absorption filter in a wavelength range of 380 to 800 nm, and Ab(λₘₐₓ) means an absorbance (maximal absorption value) at λₘₐₓ. The absorbance (maximal absorption value) and the decolorization rate at λₘₐₓ described in the column (i) of λₘₐₓ are shown in the column (i) of Ab(λₘₐₓ) and the decolorization rate, respectively. In addition, the absorbance (maximal absorption value) and the decolorization rate at λₘₐₓ described in the column (ii) of λₘₐₓ are shown in the column (ii) of Ab(λₘₐₓ) and the decolorization rate, respectively.

a*, b*: each means a tint (a*, b*) calculated by the simulation of the tint of the reflected light described above.

In the ratio of Ab(650) to Ab(λₘₐₓ) before ultraviolet irradiation, the ratio of the above-described (III) is shown in the column before ultraviolet irradiation, and the ratio of the above-described (IV) is shown in the column after ultraviolet irradiation.

### (Note in Table 3)

Transmittance: the unit is %.

Change in transmittance (T_{B}−T_{A}) by heat resistance test: a value obtained by subtracting the transmittance T_{A} after the heat resistance test from the transmittance TB before the heat resistance test, and indicating a decrease width of the transmittance due to heat of the light absorption filter (filter after UV decolorization) after ultraviolet irradiation. The unit is %.

From the results in Tables 1 to 3 above, the following facts can be seen.

In the light absorption filters Nos. c11 to c13 of the comparative examples containing the compound (6) to (9) which is a comparative azo-based coloring agent and not containing the azo-based coloring agent represented by General Formula (i), in a film decolorized by ultraviolet irradiation, the change in transmittance (decrease width of transmittance due to heat) at any of the wavelengths of 400 nm, 500 nm, and 600 nm by the heat resistance test was large.

On the other hand, in the light absorption filters Nos. 101 to 107 according to the embodiment of the present invention containing any of the compounds (1) to (5), (10), and (11) which are the azo-based coloring agents represented by General Formula (i), in a film decolorized by ultraviolet irradiation, the change in transmittance (decrease width of transmittance due to heat) at each of the wavelengths of 400 nm, 500 nm, and 600 nm by the heat resistance test was small, coloring was suppressed, and the heat resistance was excellent.

### EXPLANATION OF REFERENCES

1: upper polarizing plate
2: direction of absorption axis of upper polarizing plate
3: liquid crystal cell upper electrode substrate
4: alignment control direction of upper substrate
5: liquid crystal layer
6: liquid crystal cell lower electrode substrate
7: alignment control direction of lower substrate
8: lower polarizing plate
9: direction of absorption axis of lower polarizing plate
B: backlight unit
10: liquid crystal display device
11: aluminum foil substrate
91: light absorption filter
D65: white light having spectrum of standard illuminant D65 defined by International Commission on Illumination (CIE)
R: reflected light

What is claimed is:

1. A composition comprising:
a resin;
a compound that generates a radical upon ultraviolet irradiation; and
an azo-based coloring agent represented by General Formula (i), 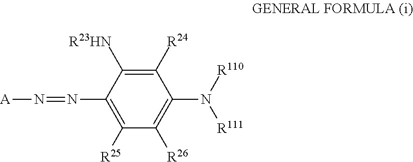
in Formula, R²³ represents a hydrogen atom, —C(═O)R¹⁰¹, —S(═O)₂R¹⁰¹, an acyclic hydrocarbon group, a monocyclic hydrocarbon group, a fused polycyclic hydrocarbon group, or a heterocyclic group,
R¹⁰¹ represents an alkyl group, an alkoxy group, an amino group, or a phenyl group,
R²⁴ to R²⁶ represent a hydrogen atom, a halogen atom, a cyano group, a nitro group, a carboxy group, a sulfo group, —OR¹⁰⁸, —SR¹⁰⁹, —NR¹¹⁰R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, or a heterocyclic group,
R¹⁰⁸ to R¹²¹ represent a hydrogen atom, an acyclic hydrocarbon group, a monocyclic hydrocarbon group, a fused polycyclic hydrocarbon group, or a heterocyclic group, n is a positive integer,
R²⁵ and R²⁶ may be linked to each other to form a hydrocarbon ring, R¹¹⁰ and R¹¹¹ may be linked to each other to form a saturated heterocyclic ring, R²³ and R²⁴ may be linked to each other to form a heterocyclic ring, R²⁴ and R¹¹⁰ may be linked to each other to form a heterocyclic ring, and R²⁶ and R¹¹¹ may be linked to each other to form a heterocyclic ring,
A represents a 5- or 6-membered aromatic hydrocarbon group or aromatic heterocyclic group.

2. The composition according to claim 1,
wherein the compound that generates the radical upon the ultraviolet irradiation includes a combination of
a compound A having an acid group, and
a compound B having a structure capable of forming a hydrogen bond with the acid group contained in the compound A.

3. The composition according to claim 2,
wherein the compound A having the acid group is chemically bonded to a polymer that constitutes the resin.

4. The composition according to claim 1,
wherein the azo-based coloring agent is decolorized by the ultraviolet irradiation.

5. A light absorption filter comprising:
a resin;
a compound that generates a radical upon ultraviolet irradiation; and
an azo-based coloring agent represented by General Formula (i), 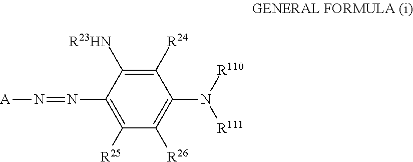
in Formula, R²³ represents a hydrogen atom, —C(═O)R¹⁰¹, —S(═O)₂R¹⁰¹, an acyclic hydrocarbon group, a monocyclic hydrocarbon group, a fused polycyclic hydrocarbon group, or a heterocyclic group,
R¹⁰¹ represents an alkyl group, an alkoxy group, an amino group, or a phenyl group,
R²⁴ to R²⁶ represent a hydrogen atom, a halogen atom, a cyano group, a nitro group, a carboxy group, a sulfo group, —OR¹⁰⁸, —SR¹⁰⁹, —NR¹¹⁰R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, or a heterocyclic group,
R¹⁰⁸ to R¹²¹ represent a hydrogen atom, an acyclic hydrocarbon group, a monocyclic hydrocarbon group, a fused polycyclic hydrocarbon group, or a heterocyclic group, n is a positive integer,
R²⁵ and R²⁶ may be linked to each other to form a hydrocarbon ring, R¹¹⁰ and R¹¹¹ may be linked to each other to form a saturated heterocyclic ring, R²³ and R²⁴ may be linked to each other to form a heterocyclic ring, R²⁴ and R¹¹⁰ may be linked to each other to form a heterocyclic ring, and R²⁶ and R¹¹¹ may be linked to each other to form a heterocyclic ring,
A represents a 5- or 6-membered aromatic hydrocarbon group or aromatic heterocyclic group.

6. The light absorption filter according to claim 5,
wherein the compound that generates the radical upon the ultraviolet irradiation includes a combination of
a compound A having an acid group, and
a compound B having a structure capable of forming a hydrogen bond with the acid group contained in the compound A.

7. The light absorption filter according to claim 6,
wherein the compound A having the acid group is chemically bonded to a polymer that constitutes the resin.

8. The light absorption filter according to claim 5,
wherein the azo-based coloring agent is decolorized by the ultraviolet irradiation.

9. An optical filter obtained by subjecting the light absorption filter according to claim 5 to mask exposure by the ultraviolet irradiation.

10. An organic electroluminescent display device, an inorganic electroluminescent display device, or a liquid crystal display device, comprising:
the optical filter according to claim 9.

11. The organic electroluminescent display device, the inorganic electroluminescent display device, or the liquid crystal display device according to claim 10,
wherein a layer that inhibits light absorption of the compound that generates the radical upon the ultraviolet irradiation is provided on a viewer side with respect to the optical filter.

12. A manufacturing method for an optical filter, comprising:
irradiating the light absorption filter according to claim 5 with an ultraviolet ray to carry out mask exposure.

13. A compound represented by General Formula (i-1), 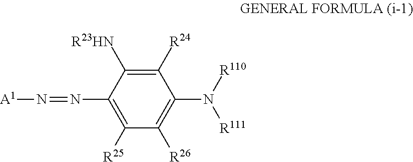
in Formula, R²³ represents a hydrogen atom, —C(═O)R¹⁰¹, —S(═O)₂R¹⁰¹, an acyclic hydrocarbon group, a monocyclic hydrocarbon group, a fused polycyclic hydrocarbon group, or a heterocyclic group,
R¹⁰¹ represents an alkyl group, an alkoxy group, an amino group, or a phenyl group,
R²⁴ to R²⁶ represent a hydrogen atom, a halogen atom, a cyano group, a nitro group, a carboxy group, a sulfo group, —OR¹⁰⁸, —SR¹⁰⁹, —NR¹¹⁰R¹¹¹, —S(═O)₂NR¹¹²R¹¹³, —C(═O)NR¹¹⁴R¹¹⁵, —NHC(═O)R¹¹⁶, —C(═O)OR¹¹⁷, —O(CH₂CH₂O)ₙR¹¹⁸, —O(CH₂CH₂S)ₙR¹¹⁹, —S(CH₂CH₂O)ₙR¹²⁰, —S(CH₂CH₂S)ₙR¹²¹, an acyclic hydrocarbon group, a monocyclic hydrocarbon group, a fused polycyclic hydrocarbon group, or a heterocyclic group,
R¹⁰⁸ to R¹²¹ represent a hydrogen atom, an acyclic hydrocarbon group, a monocyclic hydrocarbon group, a fused polycyclic hydrocarbon group, or a heterocyclic group, n is a positive integer,
R²⁵ and R²⁶ may be linked to each other to form a hydrocarbon ring, R¹¹⁰ and R¹¹¹ may be linked to each other to form a saturated heterocyclic ring, R²³ and R²⁴ may be linked to each other to form a heterocyclic ring, R²⁴ and R¹¹⁰ may be linked to each other to form a heterocyclic ring, and R²⁶ and R¹¹¹ may be linked to each other to form a heterocyclic ring,
A¹ represents a group represented by any of Formulae (A1) to (A3), 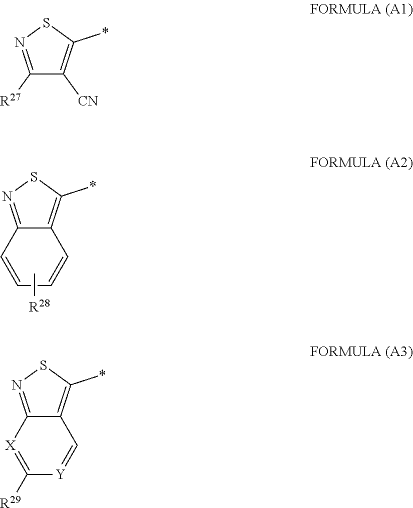
in Formula, R²⁷ represents an unsubstituted methyl group or an aromatic ring group,
R²⁸ represents a hydrogen atom, a methanesulfonyl group, or a sulfo group, or a sulfamoyl group which may be substituted with an alkyl group,
R²⁹ represents an alkyl group, a cyano group, or an alkylsulfanyl group,
X and Y represent CH or a nitrogen atom,
* represents a bonding site.
